(19) United States
(12) Patent Application Publication
(10) Pub. No.: US 2010/0202964 A1
(43) Pub. Date: Aug. 12, 2010

(54) Organ Homing Genes of Cancer Metastases

(75) Inventor: Anton Wellstein, Washington, DC (US)

Correspondence Address: WOLF GREENFIELD & SACKS, P.C., 600 ATLANTIC AVENUE, BOSTON, MA, 02210-2206, US

(73) Assignee: Georgetown University, Washington, DC (US)

(21) Appl. No.: 11/887,867

(22) PCT Filed: Apr. 5, 2006
(86) PCT No.: PCT/US2006/012797

§ 371 Date: Jun. 19, 2009

Related U.S. Application Data
(60) Provisional application No. 60/668,845, filed on Apr. 5, 2005.

(51) Int. Cl. A61K 51/00; C12Q 1/68; G01N 33/53; C07K 16/18; A61K 38/16; A61K 49/00

(52) U.S. Cl. 424/1.69; 435/6; 435/7.1; 530/387.1; 530/388.85; 514/12; 424/9.1

(57) ABSTRACT

Provided herein are methods that use liver targeting domains of colon cancer, such as for example PA28alpha, HNRNPA1, and/or KIAA1407, and the polynucleotides that encode them, for the prognosis, diagnosis, detection, staging and/or prediction of survival rates in individuals with colon cancer, whether the cancer has metastasized or not, as well as for imaging purposes and design of diagnostic and/or therapeutic methods.

### CROSS-REFERENCE TO RELATED APPLICATIONS

This application claims the benefit of U.S. Provisional Application Ser. No. 60/668,845, entitled, “Organ Homing Genes of Cancer Metastases” and filed Apr. 5, 2005. The teachings of the referenced application are incorporated herein by reference.

### STATEMENT REGARDING FEDERALLY SPONSORED RESEARCH OR DEVELOPMENT

This invention was made with government support under National Institutes of Health (NIH) SPORE Grant No. CA58185 and NIH Grant No. K04. The United States Government has certain rights in the invention.

### BACKGROUND OF THE INVENTION

Cancer is a leading cause of death in the United States. If cancers were diagnosed early in their development, such as at a localized stage, individuals would have a better chance of long term survival than is the case when diagnosis does not occur until after metastasis has occurred.

It has been clinically noted that particular primary tumors tend to metastasize to specific distant “target” organs. For example, prostate cancer often metastasizes to the bone, breast cancer may metastasize to the liver, melanomas tend to spread to lymph nodes, ovarian cancer metastasizes to other areas of the body including the lungs, brain, lymph and bones. Once a cancer has metastasized, an individual's chance of survival typically decreases. The physiologic basis for this targeting remains unknown, and neither the native homing molecules on the metastatic cell or the target molecule at the site of the secondary tumor have been identified, with singular exceptions.

In spite of advances in the diagnosis and treatment of colon cancer, there remains a well recognized need for better diagnostic and prognostic methods.

### SUMMARY OF THE INVENTION

The present invention relates to liver targeting genes of human colon cancer cells (also referred to as liver homing genes) and portions thereof; liver targeting gene products (also referred to as liver targeting proteins), homing domains of liver targeting gene products, and their uses, such as in methods of diagnosing or aiding in the diagnosis of colon cancer; methods of predicting the likelihood that a primary colon cancer will metastasize to the liver in an individual; methods of predicting or aiding in the prediction of survival of an individual after diagnosis of colon cancer (e.g., predicting or aiding in the prediction of the length of time, such as the number of years, that an individual with colon cancer will survive); methods of assessing the effectiveness of therapy; and methods of assessing the stage or progression of colon cancer in an individual. The invention further relates to the use of liver targeting genes and the encoded products for delivering agents, such as prophylactic or therapeutic drugs and detection or imaging agents, to the liver; compositions, such as pharmaceutical compositions and compositions useful for detection and/or imaging of cancer cells and tumors, which comprise a (at least one) liver targeting gene or portion thereof or an (at least one) encoded product, such as a protein or homing domain of a liver targeting gene; and antibodies that specifically bind liver targeting gene products or portions thereof, such as antibodies that specifically bind homing domains of liver targeting gene products.

In specific embodiments, the present invention relates to liver targeting genes PA28alpha, HNRNPA1 and KIAA1407, described herein; the products (e.g., proteins, also referred to as polypeptides), including homing domains, encoded by PA28alpha, HNRNPA1 or KIAA1407 or portions thereof; methods in which one or more of liver targeting genes PA28alpha, HNRNPA1 and/or KIAA1407, or portions thereof are used; methods in which one or more of the encoded products are used; compositions comprising one or more of the liver targeting genes (PA28alpha, HNRNPA1 and/or KIAA1407) or a portion(s) thereof; compositions comprising (a) a homing domain of a liver targeting polypeptide (a homing domain of PA28alpha, HNRNPA1 or KIAA1407 and (b) an agent to be targeted to colon cancer in an individual; and antibodies that specifically bind (recognize) products (proteins, polypeptides), including homing domains, encoded by PA28alpha, HNRNPA1 or KIAA1407 or portions of any of these genes. In specific embodiments, compositions of the present invention are pharmaceutical compositions that comprise (a) one or more of these liver targeting genes or portions thereof (PA28alpha, HNRNPA1 and/or KIAA1407) or one or more encoded products, such as one or more protein or one or more homing domain (e.g., one or more homing domain of PA28alpha, HNRNPA1 and/or KIAA1407) and (b) a therapeutic agent (a drug) to be delivered to the colon cancer. Therapeutic agents that can be included in a pharmaceutical composition of the present invention include those, such as ricin, radioisotopes, clotting agents, thrombolytic factors, chemotherapeutic and radiosensitizing agents, anti-angiogenesis agents, anti-motility agents, and immunomodulatory agents, useful to treat colon cancer or prevent the extent to which it occurs in an individual. In further specific embodiments, the compositions of the present invention are useful for detection and/or imaging of colon cancer (primary or after metastasis) and cancer cells derived from colon cancer (e.g., cancer cells from a primary colon cancer/tumor which are present in the blood or other body fluid or a site, such as the liver, other than the primary site).

Provided herein are compositions and methods for determining the presence of and/or the presence of elevated levels of one or more of PA28alpha, HNRNPA1, and/or KIAA1407, or polynucleotides encoding them, in a sample from an individual at risk for or subject to cancer, such as colon cancer, or at risk for or subject to metastasis, wherein the presence of or presence of elevated levels of at least one of the following liver targeting gene products: PA28alpha, HNRNPA1, KIAA1407, relative to a control, such as a population control, non-colon cancer sample, non-metastasis sample, or a non-cancer sample, a temporal sample (e.g., a sample taken from the same subject at an earlier time point), or other appropriate control(s), or polynucleotides encoding them, is correlated with increased risk of metastasis, such as metastasis to the liver. In some examples, the presence of elevated levels of one of PA28alpha, HNRNPA1, and KIAA1407, or polynucleotides encoding one of them, in a sample is correlated with an increased risk of metastasis. In some examples, the presence of elevated levels of PA28alpha and HNRNPA1; PA28alpha and KIAA1407 or HNRNPA1 and KIAA1407, or polynucleotides encoding them, in a sample is correlated with an increased risk of metastasis. In some examples, the presence of elevated levels of PA28alpha, HNRNPA1 and KIAA1407, or polynucleotides encoding the three in a sample is correlated with an increased risk of metastasis. In some examples, polynucleotides encoding PA28alpha, HNRNPA1, and KIAA1407 are shown in FIGS. 2A-2C. In other examples, polypeptides comprising targeting domains of PA28alpha, HNRNPA1, and KIAA1407 are shown in FIGS. 3A-3E as well as in FIG. 10 through FIG. 13.

Also provided herein are methods for determining the risk of metastasis in an individual diagnosed with cancer, such as for example, colon cancer; comprising detecting the presence of and/or detecting or measuring the levels of one or more of PA28alpha, HNRNPA1, and/or KIAA1407, or polynucleotides encoding them, in a sample from the individual, wherein the presence of and/or presence of elevated levels of one or more of PA28alpha, HNRNPA1, and/or KIAA1407, or polynucleotides encoding them (relative to a control) is correlated with an increased risk of metastasis. In some examples, the presence of elevated levels of any one of PA28alpha, HNRNPA1, and KIAA1407, or polynucleotides encoding one of them, in a sample, is correlated with an increased risk of metastasis. In some examples, the presence of elevated levels of PA28alpha and HNRNPA1; PA28alpha and KIAA1407; or HNRNPA1 and KIAA1407, or polynucleotides encoding them, in a sample is correlated with an increased risk of metastasis. In some examples, the presence of elevated levels of PA28alpha, HNRNPA1, and KIAA1407, or polynucleotides encoding them, in a sample is correlated with an increased risk of metastasis. In some examples, polynucleotides encoding PA28alpha, HNRNPA1, and KIAA1407 are shown in FIGS. 2A-2C. In other examples, polypeptides comprising targeting domains of PA28alpha, HNRNPA1, and KIAA1407 are shown in FIGS. 3A-3E as well as in FIG. 10 through FIG. 13.

Also provided herein are methods for determining survival rates (e.g., the probability of survival) in an individual diagnosed with colon cancer, whether or not the cancer has metastasized to a secondary tissue source, such as for example, the liver, comprising measuring the presence of and/or the presence of elevated levels of one or more of the following: 1) a polynucleotide disclosed herein in FIGS. 2A-2C, or a complement thereof; 2) a polynucleotide encoding any one of the polypeptides disclosed herein in FIGS. 3A-3E, or a complement thereof; 3) a polynucleotide encoding any one of the polypeptides disclosed herein in FIGS. 10 through 13, or a complement thereof; 4) a polypeptide disclosed herein in FIGS. 3A-3E, and/or a fragment thereof; or 5) a polypeptide disclosed herein in FIGS. 10-13, or fragment thereof, wherein the presence of or presence of elevated levels of anyone of 1) through 5) as compared to a control, is correlated with a decrease in survival rates. Provided herein are methods for determining survival rates in an individual diagnosed with colon cancer, whether or not the cancer has metastasized to a secondary tissue source, such as for example, the liver, comprising measuring the presence of and/or the presence of elevated levels of two or more of PA28alpha, HNRNPA1, and KIAA1407, or polynucleotides encoding them. In some examples, the presence of or the presence of elevated levels of PA28alpha, HNRNPA1, and KIAA1407, or polynucleotides encoding them, is correlated with a decrease in survival rates. In some examples, the presence of elevated cumulative levels of PA28alpha, HNRNPA1, and KIAA1407, that is, the additive measurement of PA28alpha, HNRNPA1, and KIAA1407 together, is correlated with a decrease in survival rates (e.g., correlated with an increase in mortality rates), and in some examples, is correlated with a decrease in five year and ten year survival rates. Such methods comprise measuring any one, two or three of PA28alpha, HNRNPA1, and/or KIAA1407, or polynucleotides encoding them, alone or together with any one or more of the other liver homing domains disclosed herein in FIGS. 3A-3E, or FIGS. 10-13, or polynucleotides encoding them, such as the polynucleotides disclosed in FIGS. 2A-2C in samples from individuals at risk for or subject to cancer, or at risk for or subject to recurrence of cancer, or at risk for or subject to metastasis. In some examples, the samples are biopsy samples.

Also provided herein are compositions comprising such polynucleotides and polypeptides, as well as kits comprising such polynucleotides and polypeptides. Provided herein are sets of reagents specific for any of PA28alpha, HNRNPA1 and KIAA1407, or polynucleotides encoding them, or specific for any combination of PA28alpha, HNRNPA1, and KIAA1407 (PA28alpha and HNRNPA1; PA28alpha and KIAA1407; HNRNPA1 and KIAA1407), or polynucleotides encoding them, for use in the methods disclosed herein. Also provided herein are diagnostic and therapeutic methods.

In certain embodiments, the present invention provides a method of predicting the likelihood that a primary colon cancer in an individual will metastasize to the liver of the individual. Such method comprises analyzing the primary colon cancer for at least one of the following: (a) a liver targeting gene or a complement thereof; (b) a protein encoded by a liver targeting gene; (c) a homing domain of a liver targeting gene product; (d) nucleic acid encoding a homing domain of (c) or a complement of the nucleic acid encoding a homing domain of (c); and (e) mRNA of a horning domain of (c) or the complement thereof, wherein if at least one of (a)-(e) is present in the primary colon cancer, there is an increased likelihood the primary colon cancer will metastasize to the liver of the individual than if at least one of (a)-(e) is present at a lower level relative to an appropriate control. Optionally, the liver targeting gene is selected from: (i) a gene encoding PA28 alpha; (ii) a gene encoding HNRNP A1; and (iii) a gene encoding KIAA1407.

Optionally, the homing domain of a liver targeting gene product is selected from: (i) a homing domain of PA28 alpha; (ii) a homing domain of HNRNP A1; and (iii) a homing domain of KIAA1407. Optionally, the nucleic acid encoding a homing domain is selected from: (i) nucleic acid encoding a homing domain of PA28 alpha; (ii) nucleic acid encoding a homing domain of HNRNP A1; and (iii) nucleic acid encoding a homing domain of KIAA1407. Optionally, the mRNA of a homing domain is selected from (i) mRNA of a homing domain of PA28 alpha; (ii) mRNA of a homing domain of HNRNP A1; and (iii) mRNA of a homing domain of KIAA1407. For example, the homing domain of a liver targeting gene product includes, but is not limited to, a C-terminal portion of PA28 alpha; a central portion of HNRNP A1, and an N-terminal region of KIAA1407. To illustrate, the C-terminal portion of PA28 alpha is encoded by SEQ ID NO: 7; the central portion of HNRNP A1 is encoded by SEQ ID NO: 5; and the N-terminal region of KIAA1407 is encoded by SEQ ID NO: 8. In certain cases, the primary colon cancer is analyzed for one or more of the following: (i) PA28 alpha; (ii) a homing domain of PA28 alpha; (iii) HNRNP A1; (iv) a homing domain of HNRNP A1; (v) KIAA1407 and (vi) a homing domain of KIAA1407.

In certain cases, at least one antibody that specifically recognizes (i) PA28 alpha; (ii) a homing domain of PA28 alpha; (iii) HNRNP A1; (iv) a homing domain of HNRNP A1; (v) KIAA1407 or (vi) a homing domain of KIAA1407 is used to analyze the primary colon cancer. For example, a combination comprising at least two of the following antibodies: (a) an antibody that specifically recognizes PA28 alpha; (b) an antibody that specifically recognizes HNRNP A1; and (c) an antibody that specifically recognizes KIAA1407 is used to analyze the primary colon cancer. Alternatively, a combination comprising at least two of the following antibodies: (a) an antibody that specifically recognizes a homing domain of PA28 alpha; (b) an antibody that specifically recognizes a homing domain of HNRNP A1; and (c) an antibody that specifically recognizes a homing domain of KIAA1407 is used to analyze the primary colon cancer.

In certain cases, the primary colon cancer is analyzed for one or more of the following: (a) a gene encoding PA28 alpha or a complement thereof; (b) a gene encoding HNRNP A1 or a complement thereof; (c) a gene encoding KIAA1407 or a complement thereof; (d) nucleic acid encoding a homing domain of PA28 alpha or a complement thereof; (e) nucleic acid encoding a homing domain of HNRNP A1 or a complement thereof; (f) nucleic acid encoding a homing domain of KIAA1407 or a complement thereof; (g) mRNA of a homing domain of PA28 alpha or a complement thereof; mRNA of a homing domain of HNRNP A1 or a complement thereof; and (iii) mRNA of a homing domain of KIAA1407 or a complement thereof. Optionally, the primary colon cancer is analyzed for mRNA expression of a liver targeting gene or mRNA expression of a homing domain of a liver targeting gene. For example, the primary colon cancer is analyzed by in situ hybridization for mRNA expression of at least one of the following: (a) a gene encoding PA28 alpha or a complement thereof; (b) a gene encoding HNRNP A1 or a complement thereof; (c) a gene encoding KIAA1407 or a complement thereof; (d) nucleic acid encoding a homing domain of PA28 alpha or a complement thereof; (e) nucleic acid encoding a homing domain of HNRNP A1 or a complement thereof; (f) nucleic acid encoding a homing domain of KIAA1407 or a complement thereof; (g) mRNA of a homing domain of PA28 alpha or a complement thereof; (h) mRNA of a homing domain of HNRNP A1 or a complement thereof; and (i) mRNA of a homing domain of KIAA1407 or a complement thereof.

In certain embodiments, the present invention provides a method of predicting or aiding in the prediction that an individual with colon cancer will survive at least one year after diagnosis of a primary colon cancer. Such method comprises (a) analyzing a colon cancer sample obtained from the individual for expression of at least one liver targeting gene, thereby producing a sample score for the individual; (b) comparing the sample score for the individual with results of corresponding analysis, referred to as a control score, of colon cancer samples obtained from individuals who survived at least one year after diagnosis of a primary colon cancer, wherein if the sample score for the individual is greater than the control score, the individual with colon cancer is less likely to survive at least one year after diagnosis than if the sample score for the individual is less than the control score.

Optionally, the colon cancer sample is selected from a sample from a primary colon cancer, a sample from a tissue to which the primary colon cancer has metastasized, a body fluid (e.g., blood, serum, plasma, a blood-derived fraction, stool, colonic effluent or urine). In certain cases, the colon cancer sample is analyzed for at least one liver targeting gene; at least one homing domain of a liver targeting gene; at least one nucleic acid encoding a homing domain of a liver targeting gene; or at least one mRNA of a homing domain of a liver targeting gene, thereby producing a sample score for the individual. Optionally, the sample score is compared with results of corresponding analysis of colon cancer samples obtained from individuals who survived at least one year after diagnosis of a primary colon cancer.

For example, (a) the liver targeting gene is a gene encoding PA28 alpha or a complement thereof; a gene encoding HNRNP A1 or a complement thereof; a gene encoding KIAA1407 or a complement thereof; (b) the homing domain of a liver targeting gene is a homing domain of PA28 alpha; a homing domain of HNRNP A1; or a homing domain of KIAA1407; (c) the nucleic acid encoding a homing domain of a liver targeting gene is a nucleic acid encoding a homing domain of PA28 alpha; a nucleic acid encoding a homing domain of HNRNP A1; or nucleic acid encoding a homing domain of KIAA1407; and (d) the mRNA of a homing domain of a liver targeting gene is mRNA of a homing domain of PA28 alpha; mRNA of a homing domain of HNRNP A1; or mRNA of a homing domain of KIAA1407.

In certain cases, the colon cancer sample is analyzed for at least two or three liver targeting genes; a homing domain of at least two or three liver targeting gene products; at least two or three nucleic acids, each of which encodes a homing domain of a different liver targeting gene; or at least two or three mRNAs, each of which is a homing domain of a different liver targeting gene product, thereby producing a sample score for the individual. Optionally, the sample score is compared with results of corresponding analysis of colon cancer samples obtained from individuals who survived at least one year after diagnosis of a primary colon cancer. To illustrate, the method involves analyzing a colon cancer sample obtained from the individual for expression of at least two of the following liver targeting genes: PA28 alpha; HNRNP A1; and KIAA1407, thereby producing a sample score for the individual and comparing the sample score for the individual with results of corresponding analysis, referred to as a control score, of colon cancer samples obtained from individuals who survived at least one year after diagnosis of a primary colon cancer. In certain cases, the method comprises analyzing a colon cancer sample obtained from the individual for expression of PA28 alpha; HNRNP A1; and KIAA1407, thereby producing a sample score for the individual and comparing the sample score for the individual with results of corresponding analysis, referred to as a control score, of colon cancer samples obtained from individuals who survived at least one year after diagnosis of a primary colon cancer. Optionally, the method is for predicting or aiding in predicting that the individual will survive at least 5 years after diagnosis.

In certain embodiments, the present invention provides a method of targeting an agent to colon cancer in an individual, comprising administering to an individual in need thereof an effective amount of a composition comprising a homing domain of a liver targeting polypeptide and the agent to be targeted to colon cancer in the individual, whereby the agent is targeted to the colon cancer. For example, the agent to be targeted to colon cancer is a therapeutic drug or a detection agent and the colon cancer is a primary colon cancer or colon cancer that has metastasized to the liver. To illustrate, the agent is a therapeutic drug selected from ricin, a radioisotope, a chemotherapeutic and radiosensitizing agent, an anti-angiogenesis agent, an anti-motility agent, and an immunomodulatory agent. In certain cases, the agent is a detection agent such as radioisotopes, dyes, fluorescent molecules, and pigments.

In certain embodiments, the present invention provides an isolated antibody which specifically binds PA28alpha, HNRNPA1 or KIAA1407. Optionally, the antibody specifically binds a homing domain of PA28alpha, HNRNPA1 or KIAA1407. In certain cases, the antibody is a monoclonal antibody.

In certain embodiments, the present invention provides a composition which comprises (a) a homing domain of a liver targeting polypeptide, and (b) an agent to be targeted to colon cancer in an individual. For example, the homing domain is a homing domain of PA28alpha; a homing domain of HNRNPA1; or a homing domain of KIAA1407, and the agent to be targeted to colon cancer is a therapeutic drug (e.g., ricin, a radioisotope, a chemotherapeutic and radiosensitizing agent, an anti-angiogenesis agent, an anti-motility agent, and an immunomodulatory agent) or a detection agent (e.g., a radioisotope, a dye, a fluorescent molecule, a pigment). Optionally, the subject composition is a pharmaceutical composition which further comprises a pharmaceutically acceptable carrier.

### BRIEF DESCRIPTION OF THE DRAWINGS

FIGS. 1A-1D show a schematic representation of the experimental protocol that provides for identification of phage inserts that encode domains that are associated with localization of colon cancer cells to the liver. FIG. 1A shows that mice were injected with the LS174T cDNA expression library over three successive rounds of biopanning using the liver-retained clones. FIG. 1B is a schematic representation of in vivo selection of organ-selective phage inserts. FIGS. 1C and 1D show phage titers after each round of biopanning. Corresponding PCR gels created using T7 Up and Down arms as primers reveal band clarification after the second and third rounds of biopanning. FIG. 1D shows organ distribution of injected phage clones after the fourth round of biopanning.

FIGS. 2A-2C show the polynucleotide sequences for SEQ ID NOs: 1-9.

FIGS. 3A-3E show the polypeptide sequences for SEQ ID NOs: 10-36.

FIGS. 4A-4D. FIGS. 4A-4C show the frequency of detectable expression of (FIG. 4A) PA28alpha (Genbank accession no. Q06323; UniGene Cluster Hs.75348 Homo sapiens, “PA”); (FIG. 4B) HNRNPA1 (Genbank accession no. NP_{—}002127; UniGene Cluster Hs.357721 Homo sapiens, “HN”); and (FIG. 4C) protein, KIAA1407 (Genbank accession no. A45973; UniGene cluster Hs.221642 Homo sapiens, “KI”) in normal colon, invasive colon cancer (CoCa) or breast cancer (BrCa) without and with metastasis at the time of diagnosis as well as in liver metastases of colon cancers. Legend: n=number of cases. FIG. 4D shows the cumulative expression levels for PA, HN, and KI in normal colon, colon cancer and metastasis tissues. The percent of cases with the respective cumulative expression levels are shown. Normal versus cancer, p<0.001; cancer without metastasis versus with metastasis, p<0.001.

FIGS. 5A-5B show the effect of PA, HN and KI on endothelial attachment and liver homing of human metastatic colon cancer cells. LS174T cells were allowed to attach to cultured endothelial cells (HUVEC) in the presence of control or liver homing phage, that is, phage comprising polynucleotides encoding polypeptides specific for liver. FIG. 5A shows the relative number of tumor cells prevented from endothelial attachment by incubation with phage. FIG. 5B shows LS174T tumor cells transiently transfected with a luciferase expression vector were mixed with control phage, liver homing phage from round four (“all” that is, phage which encode the amino acid sequences listed in FIGS. 3A-3E) or PA28alpha (PA) containing phage and injected into the spleen as a conduit for seeding to the liver. FIG. 5B shows the ratio of tumor cells homing to the liver after 10 minutes.

FIGS. 6A-6B show the correlation of PA28alpha, HNRNP and KIAA, cumulatively (FIG. 6A) and individually (FIG. 6B) with 5 year (60 months) survival rates in individuals diagnosed with colon cancer. *, p<0.05 relative to normal colon; and **, p<0.01 relative to normal colon and to colon cancer without metastasis among the groups as indicated in the figures.

FIGS. 7A-7D show the correlation of PA28alpha, HNRNP and KIAA, cumulatively (FIG. 7A) and individually (FIG. 7B) with 5 year (60 months) survival rates in individuals diagnosed with colon cancer and cumulatively (FIG. 7C) and individually (FIG. 7D) with 10 year (120 months) survival rates in individuals diagnosed with breast cancer. *, p<0.05 relative to normal colon; and **, p<0.01 relative to normal colon and to colon cancer without metastasis among the groups as indicated in the figures.

FIG. 8 shows 5 year survival rates of Stage II Colon Cancer Patients as described in the Examples.

FIGS. 9A-9C show the protein fragments identified as the liver homing domain of the respective genes (boxed regions) relative to the full-length protein. FIG. 9A represents PA28alpha, FIG. 9B represents HNRNPA1 and FIG. 9C represents KIAA1407. Phage selected with these inserts bind tightly to the blood vessel lining of liver tissues when injected into animals.

FIG. 10 shows the amino acid sequence for HNRNPa1 (NP_{—}002127).

FIG. 11 shows the amino acid sequence for PA28alpha (Q06323). Amino acid residue 80 is marked as showing the start of a targeting domain for PA28alpha as shown in FIG. 9A (also SEQ ID NO: 30).

FIG. 12 shows the amino acid sequence for human trichohyalin (A45973).

FIG. 13 shows the amino acid sequence for KIAA1407 (BAA92645).

### DETAILED DESCRIPTION OF THE INVENTION

The inventors discovered that the presence of and/or presence of elevated levels of a liver targeting domain, or a collection of liver targeting domains as described herein, or polypeptide(s) encoding such domains, or collection of domains, of colon cancer in a sample from an individual diagnosed with colon cancer can be used as a prognostic marker, that is for example, to detect subclinical metastasis, or to detect the risk of metastasis; as a diagnostic marker, that is for example to detect metastasis, or monitor disease progression or treatment; and/or to determine survival rates, such as for example, five and ten year survival rates in the individual, whether or not the colon cancer has metastasized.

Accordingly, provided herein are compositions and methods for determining the presence of and/or the presence of elevated levels of one or more of PA28alpha, HNRNPA1, and KIAA1407, or polynucleotides encoding them, in a sample from an individual at risk for or subject to cancer, such as colon cancer, or at risk for or subject to metastasis, wherein the presence of and/or presence of elevated levels of one or more of PA28alpha, HNRNPA1, and KIAA1407 (relative to a control, such as a population control, non-colon cancer sample, non-metastasis sample, or a non-cancer sample, a temporal sample (e.g., a sample taken from the same subject at an earlier time point), or other appropriate control(s), such as the presence of both positive and negative controls), or polynucleotides encoding them, is correlated with increased risk of metastasis, such as metastasis to the liver. Also provided herein are methods for determining the presence of and/or presence of elevated levels of PA28alpha, HNRNPA1, and KIAA1407, or polynucleotides encoding them, in a sample from an individual at risk for or subject to cancer, such as colon cancer, wherein the presence of and/or elevated levels of PA28alpha, HNRNPA1, and KIAA1407, or polynucleotides encoding them, is correlated with increased risk of metastasis, such as metastasis to the liver. In some examples, the presence of cumulative elevated levels of PA28alpha, HNRNPA1, and KIAA1407, or polynucleotides encoding them (relative to a control, such as a non-colon cancer sample, or a non-cancer sample) are correlated with an increased risk of metastasis. As shown herein HNRNPA1 is JN40; PA28alpha is LS42; and KIAA1407 is LS45.

Also provided herein are compositions and methods for determining the risk of metastasis in an individual diagnosed with cancer, such as for example, colon cancer; comprising detecting the presence of and/or detecting or measuring the levels of one or more of PA28alpha, HNRNPA1, and KIAA1407, or polynucleotides encoding them, in a sample from the individual, wherein the presence of and/or presence of elevated levels of one or more of PA28alpha, HNRNPA1, and KIAA1407, or polynucleotides encoding them (relative to a control) is correlated with an increased risk of metastasis. Also provided herein are methods for determining the risk of metastasis in an individual diagnosed with cancer, such as for example, colon cancer; comprising detecting the presence of and/or detecting or measuring the levels of PA28alpha, HNRNPA1, and KIAA1407, or polynucleotides encoding them, in a sample from the individual, wherein the presence and/or presence of elevated levels of PA28alpha, HNRNPA1, and KIAA1407, or polynucleotides encoding them (relative to a control) is correlated with an increased risk of metastasis.

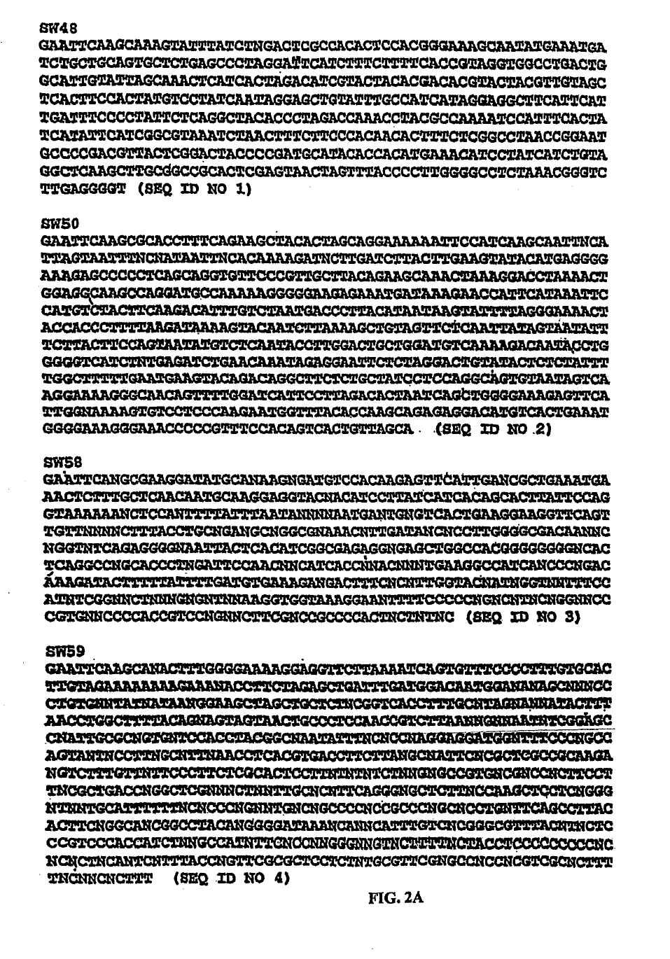

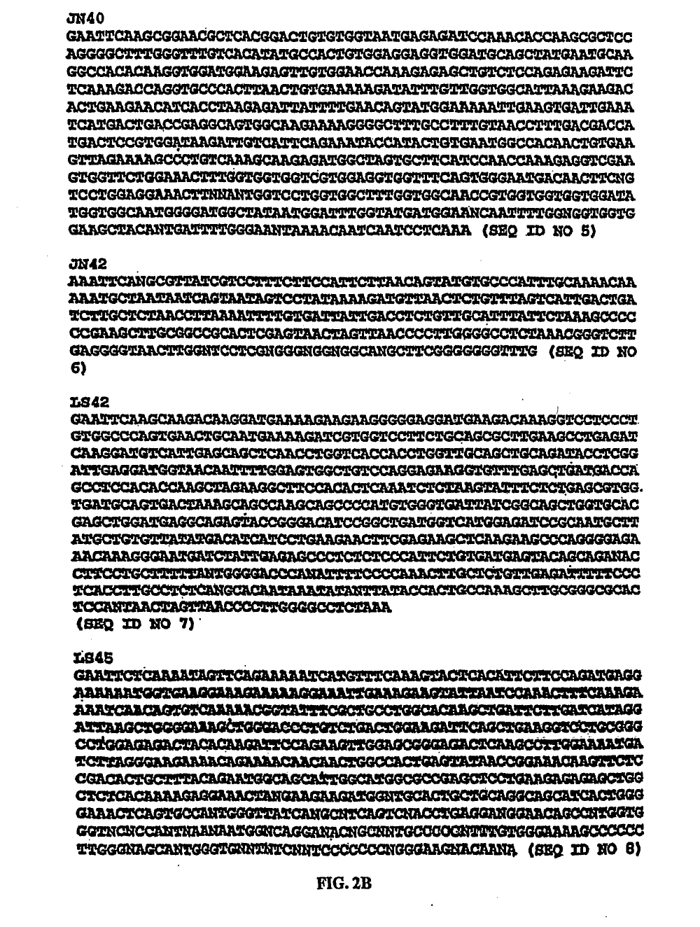

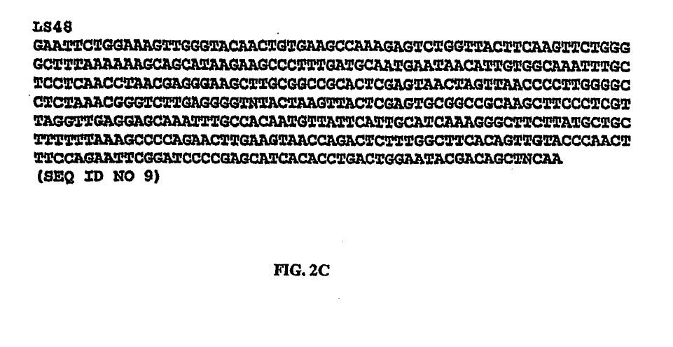

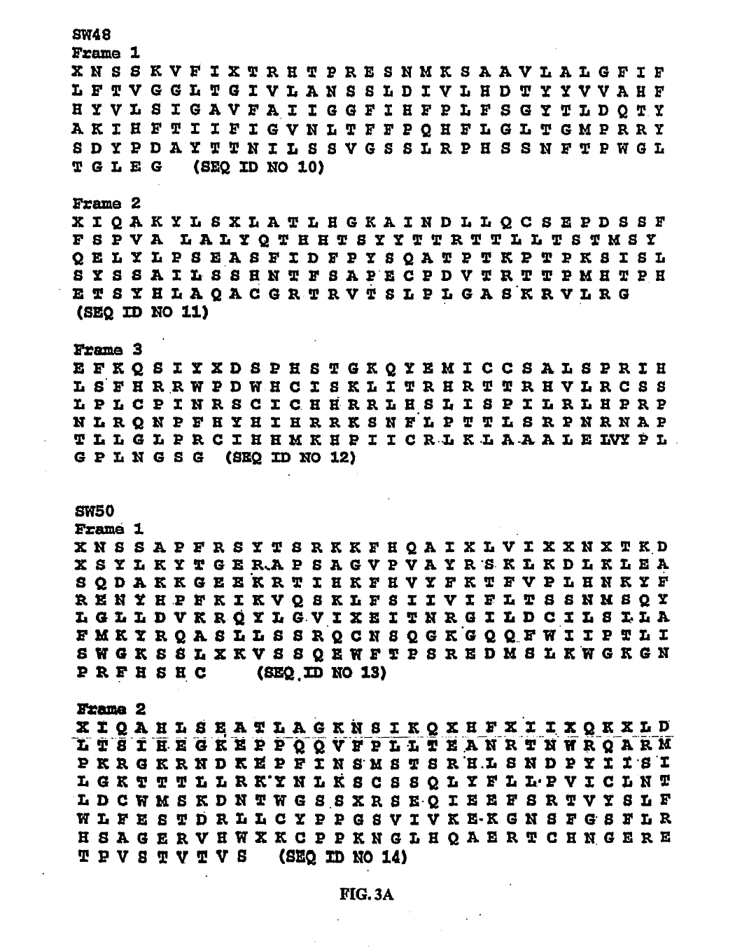

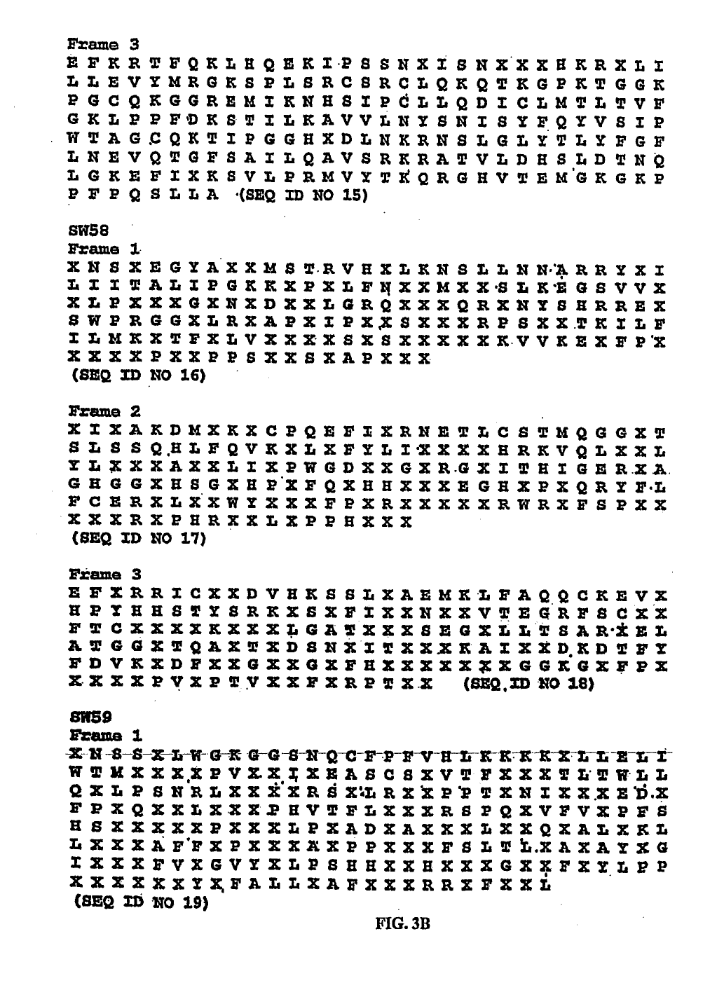

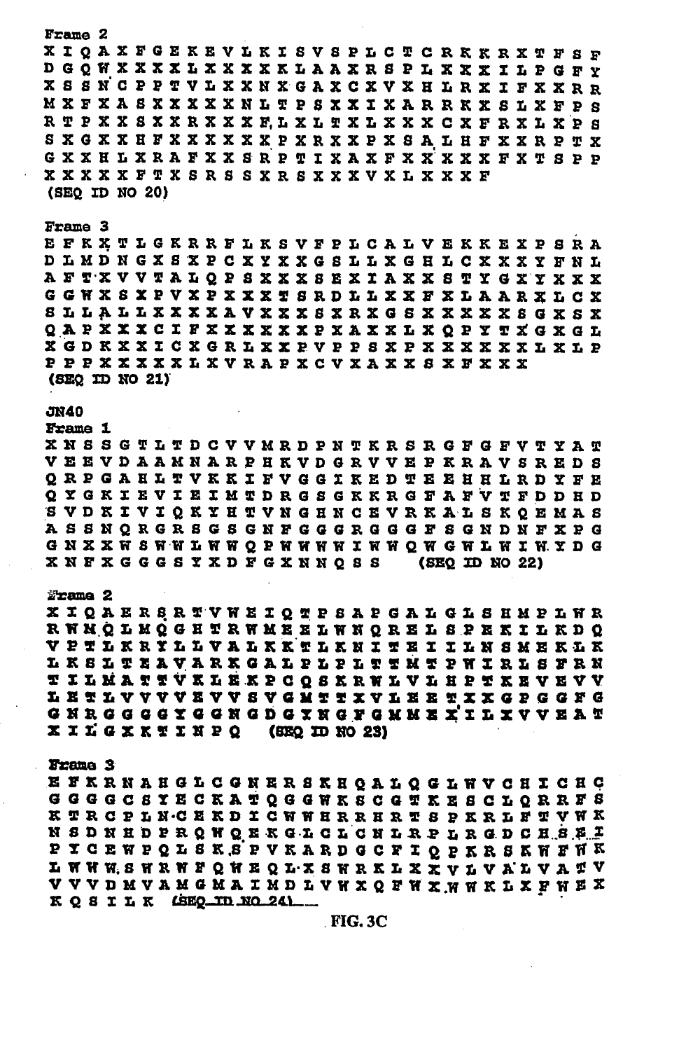

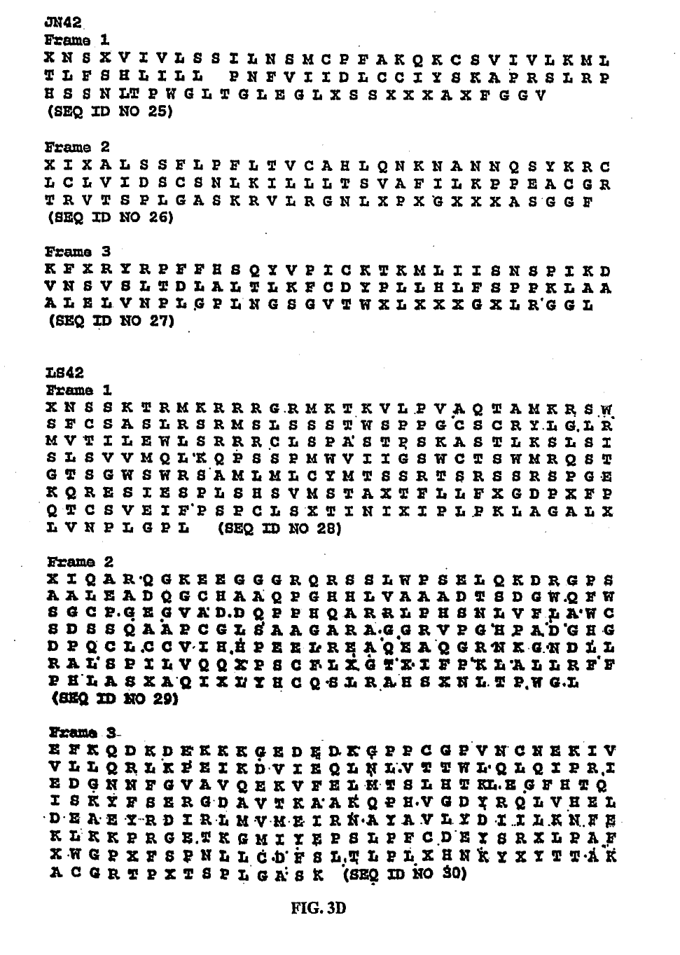

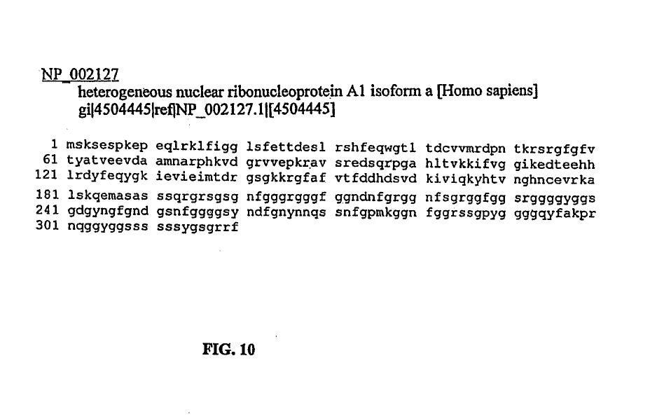

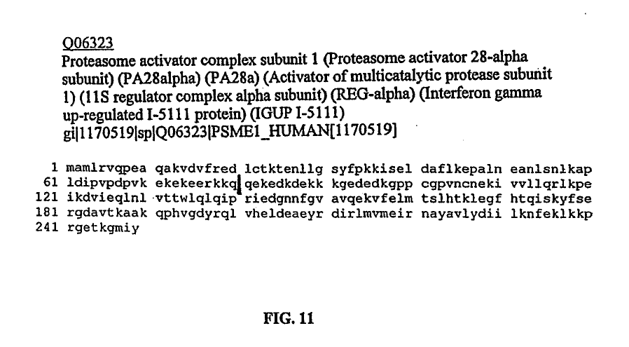

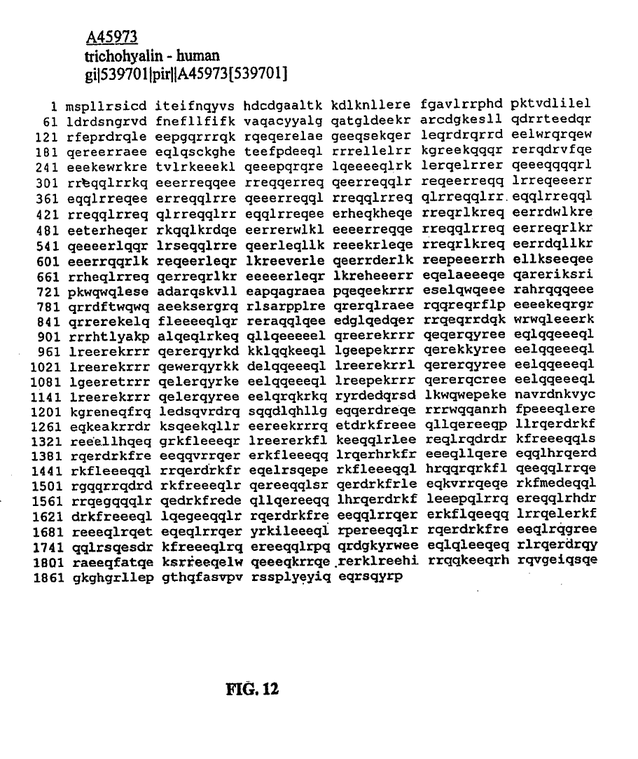

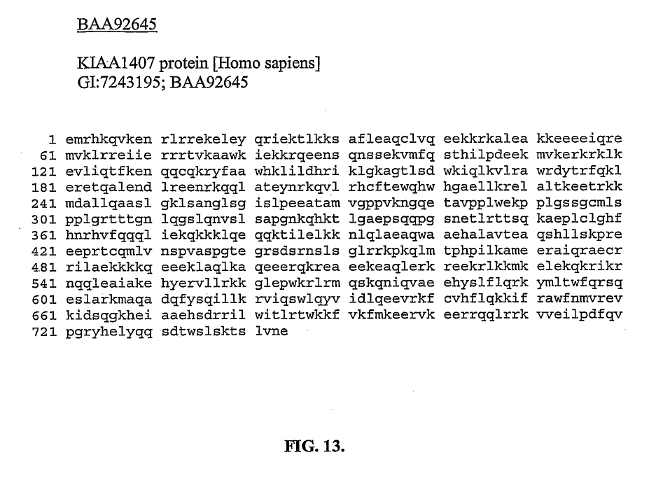

Also provided herein are compositions and methods for determining survival rates in an individual diagnosed with colon cancer, whether or not the cancer has metastasized to a secondary tissue source, such as for example, the liver, comprising detecting or measuring the presence of and/or the presence of elevated levels of one or more of the following: 1) a polynucleotide disclosed herein in FIGS. 2A-2C, or a complement thereof; 2) a polynucleotide encoding any one of the polypeptides disclosed herein in FIGS. 3A-3E, or a complement thereof; 3) a polynucleotide encoding any one of the polypeptides disclosed herein in FIGS. 10 through 13, or a complement thereof; 4) a polypeptide disclosed herein in FIGS. 3A-3E, and/or a fragment thereof ; or 5) a polypeptide disclosed herein in FIGS. 10-13, or fragment thereof, wherein the presence of or presence of elevated levels of anyone of 1) through 5) as compared to a control, is correlated with a decrease in survival rates. Provided herein are methods for determining survival rates in an individual diagnosed with colon cancer, whether or not the cancer has metastasized to a secondary tissue source, such as for example, the liver, comprising measuring the presence of and/or the presence of elevated levels of one or more of PA28alpha, HNRNPA1, and KIAA1407, or polynucleotides encoding them. Also provided herein are methods for determining survival rates in an individual diagnosed with colon cancer, whether or not the cancer has metastasized to a secondary tissue source, such as for example, the liver, comprising measuring the presence of and/or the presence of elevated levels of any one, two or three of PA28alpha, HNRNPA1, and KIAA1407, or polynucleotides encoding them (relative to a control, such as a non-colon cancer sample, or a non-cancer sample), in a sample from the individual wherein the presence of or the presence of elevated levels of any one, two or three of PA28alpha, HNRNPA1, and KIAA1407, or polynucleotides encoding them, is correlated with a decrease in survival rates. In some examples, the presence of elevated cumulative levels of PA28alpha, HNRNPA1, and KIAA1407, that is, the additive measurement of PA28alpha, HNRNPA1, and KIAA1407 together, is correlated with a decrease in survival rates (that is, correlated with an increase in mortality rates), and in some examples, is correlated with a decrease in five year and ten year survival rates. Such methods comprise measuring any one, two or three of PA28alpha, HNRNPA1, and/or KIAA1407, or polynucleotides encoding them, alone or together with any one or more of the other liver homing domains disclosed herein in FIGS. 3A-3E, or polynucleotides encoding them, such as the polynucleotides disclosed in FIGS. 2A-2C, or polypeptides disclosed herein in FIGS. 10-13, or polynucleotides encoding them, in samples from individuals in need, such as at risk for or subject to cancer or metastasis. In some examples, the samples are biopsy samples from an individual at risk for colon cancer; subject to colon cancer, wherein the cancer can be any stage of cancer including Stage I, II, III or IV; subject to recurrence of primary colon cancer; at risk for or subject to metastasis, whether or not the primary colon cancer has been surgically excised; and in some examples, the samples are collected at the time of surgical excision of the primary colon cancer. In additional examples, samples include blood, biopsy sample, pathology sample, urine or cerebrospinal fluid. Information obtained by such methods can be used to determine appropriate diagnostic and/or therapeutic intervention. As one example, since individuals with early stage, localized colon cancer that has been surgically removed have a reported 5 year mortality rate of 20% to 30%, the use of methods as disclosed herein can assist physicians in determining which individuals in early stage cancer have a high risk of metastasis and/or a high risk of decreased survival (that is, increased mortality) and design appropriate diagnostic and/or therapeutic intervention. In other examples, for individuals for whom the diagnosis of progression to Stage II or III cancer is questionable, the methods as disclosed herein can be used to determine risk of or presence of metastasis and/or risk of decreased survival rates. In another example, occult micrometastasis could be identified using PA28alpha, HNRNPA1, and KIAA1407 in imaging techniques and then treated at the time of surgery even when no metastatic disease has been otherwise identified using conventional techniques.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### DEFINITIONS

As used herein, the singular form “a”, “an”, and “the” includes plural references unless indicated otherwise. For example, “a” polynucleotide includes one or more polynucleotides and “a targeting domain” means one or more targeting domains.

The term “targeting domain” or “homing domain” or “homing molecule” or “homing protein” generally, but not exclusively, refers to a polypeptide that selectively or preferentially targets, that is binds, a particular cell type and/or tissue. By way of example, a targeting domain directs tumor cell localization from a primary tumor (e.g., colon cancer) to secondary sites (e.g., liver, lung, marrow and/or lung).

A “homing gene” or “horning signature gene” refers to a polynucleotide encoding a horning or targeting domain as described herein.

The term “selectively targets” or “preferentially targets” (used interchangeably herein) is a term well understood in the art, and methods to determine such specific or preferential targeting are also well known in the art. A polypeptide is said to exhibit “selective” or “preferential” targeting if it reacts or associates more frequently, more rapidly, with greater duration and/or with greater affinity with a particular cell and/or tissue than it does with alternative cells and/or tissues. “Selectively targets” or “preferentially targets” does not necessarily require (although it can include) exclusive binding. By way of example, a polypeptide identified by the methods described herein selectively or preferentially targets a cell or tissue if it exhibits between about 3 to about 300 fold selectivity. Methods for determining selective or preferential targeting are exemplified herein.

A “subject” or “individual” may be any animal, preferably a vertebrate, most preferably a mammal such as a human. Examples include, but are not limited to, rodents (e.g., mouse or rats), cats, dogs, rabbits, farm animals (e.g., pigs, horses, cows) or humans.

As embodied and broadly described herein, the present invention is directed to the use of liver targeting domains of colon cancer. In some examples, the targeting domains are used for the detection, diagnosis, prognosis, staging and/or prediction of survival rates of liver cancer metastasis as well as for imaging purposes and design of therapeutic methods.

Methods of Use

Colorectal cancer is the second leading cause of cancer deaths in the United States. 20% of the individuals subject to colon cancer present with liver metastasis, and 35% to 40% of potentially cured individuals will recur with local or metastatic disease with 90% of the recurrences occurring in the first 3 years. Early stage colon cancers that appear to be only local and appear to not have spread to distant sites still have a reported 5-year mortality rate of 20% to 30%. Without being bound by theory, this appears to be due, at least in part, to the re-growth of micrometastases that were not detectable at the time of diagnosis or surgical removal of the primary tumor. In individuals with Stage II or Stage III colon cancer, it is critical to identify metastatic disease so that appropriate diagnostic and/or therapeutic intervention can be determined.

The inventors discovered that the presence of or the presence of elevated levels of a liver targeting domain, or a collection of liver targeting domains as described herein, or polynucleotide(s) encoding such domains, of colon cancer disclosed herein in a sample from an individual, and in some examples, an individual diagnosed with colon cancer, can be used as a prognostic marker, that is for example, to detect subclinical metastasis, or to detect the risk of metastasis; as a diagnostic marker, that is for example to detect metastasis, or monitor disease progression or treatment; and/or to determine survival rates, such as for example, five and ten year survival rates in the individual, whether or not the colon cancer has metastasized. The present inventors obtained tissue samples from individuals with: 1) colon cancer metastasis to the liver; 2) primary colon cancers that had metastasized at the time of diagnosis; 3) invasive colon cancers without known metastasis; and all were compared with samples from normal colon tissues and samples from individuals with breast cancer due to the relatively low propensity of breast cancer for initial metastasis to the liver. The samples were analyzed for the expression of identified liver targeting domains, PA28alpha (Genbank accession no. Q06323; UniGene Cluster Hs.75348 Homo sapiens, “PA”); HNRNPA1 (Genbank accession no. NP_{—}002127; UniGene Cluster Hs.357721 Homo sapiens, “HN”); and KIAA1407 (Genbank accession no. A45973; UniGene cluster Hs.221642 Homo sapiens, “KI”). As shown herein HNRNPA1 is JN40; PA28alpha is LS42; and KIAA1407 is LS45. The analysis was carried out with tissue microarrays and included >1200 tissue samples for each of the genes studied. The results showed that 60% of invasive colon cancer without metastasis and >95% of the samples from metastatic cancers showed expression of all three of the following identified liver targeting domains, PA28alpha; HNRNPA1; and KIAA1407; whereas less than 10% of normal colon tissues showed any detectable signal. Furthermore, a significant difference in the frequency of expression was observed between cancer and normal tissues and between non-metastatic and metastatic colon cancer samples. Invasive breast cancers used as another control showed an overall low frequency of expression of the liver homing genes and no difference with respect to the metastatic phenotype.

Accordingly, provided herein are methods for determining the presence of PA28alpha, HNRNPA1, and KIAA1407, or polynucleotides encoding them, in a sample from an individual at risk for or subject to cancer, such as colon cancer, wherein the presence of PA28alpha, HNRNPA1, and KIAA1407, or polynucleotides encoding them, is correlated with increased risk of metastasis, such as metastasis to the liver. In some examples, the presence of elevated levels of PA28alpha, HNRNPA1, and KIAA1407, or polynucleotides encoding them, including cumulative elevated levels, is correlated with an increased risk of metastasis and a decrease in survival rates (that is, an increase in mortality rates). Also provided herein are methods for determining the risk of metastasis in an individual diagnosed with cancer, such as for example, colon cancer; comprising detecting the presence of and/or measuring the levels of any one, two or three of PA28alpha, HNRNPA1, and KIAA1407, or polynucleotides encoding them, in a sample from the individual, wherein the presence and/or presence of elevated levels of any one, two or three of PA28alpha, HNRNPA1, and KIAA1407, or polynucleotides encoding them, is correlated with an increased risk of metastasis and a decrease in survival rates.

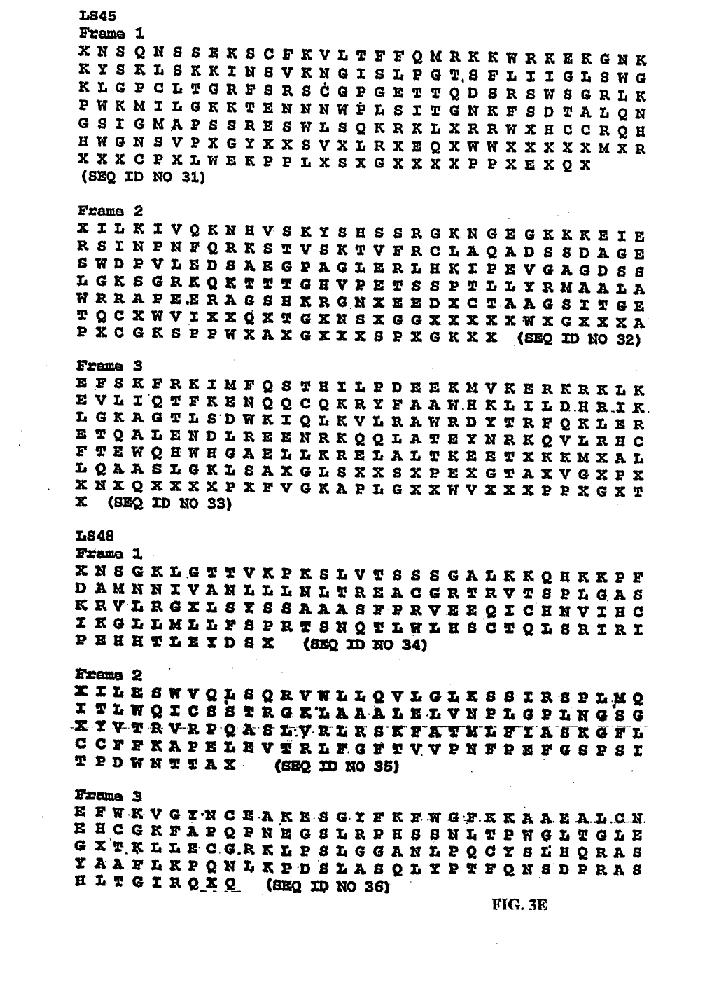

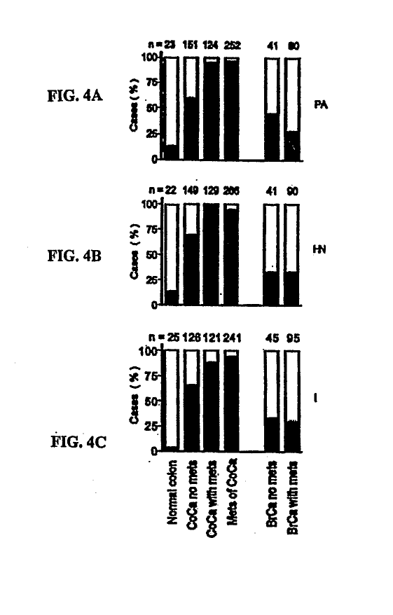

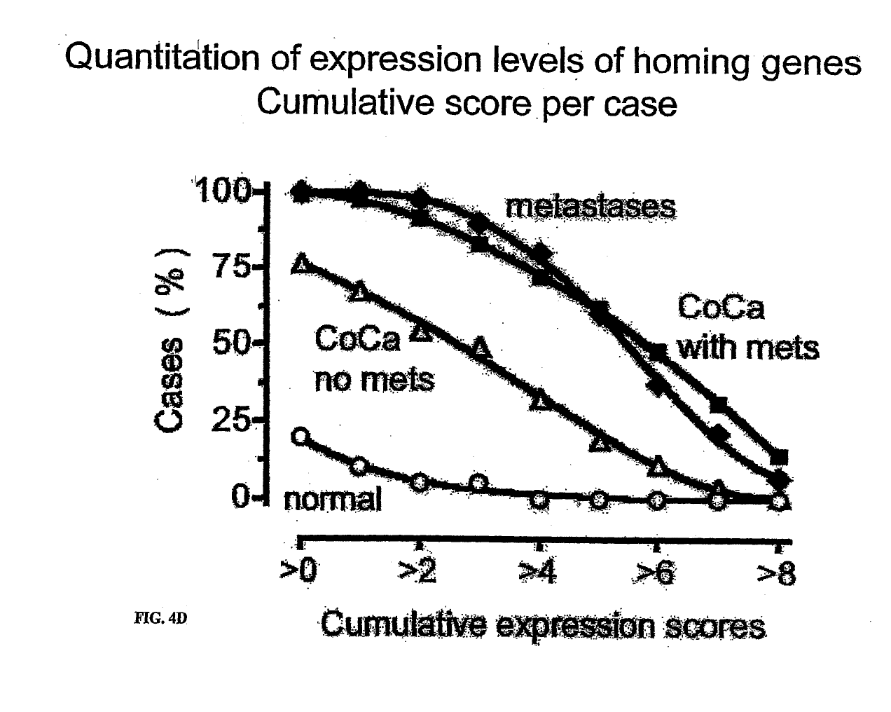

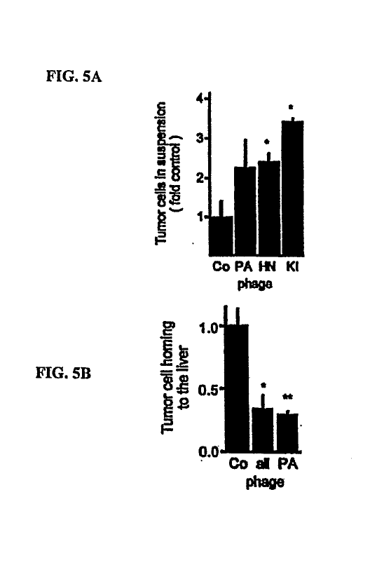

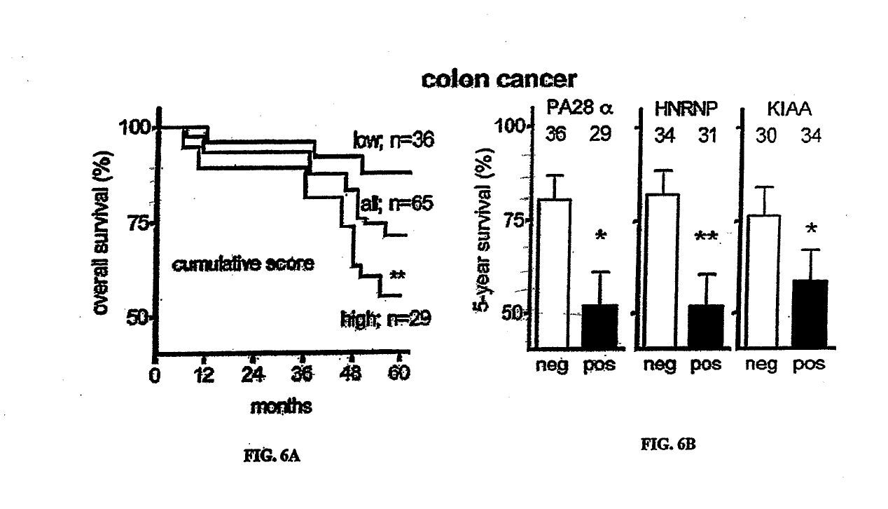

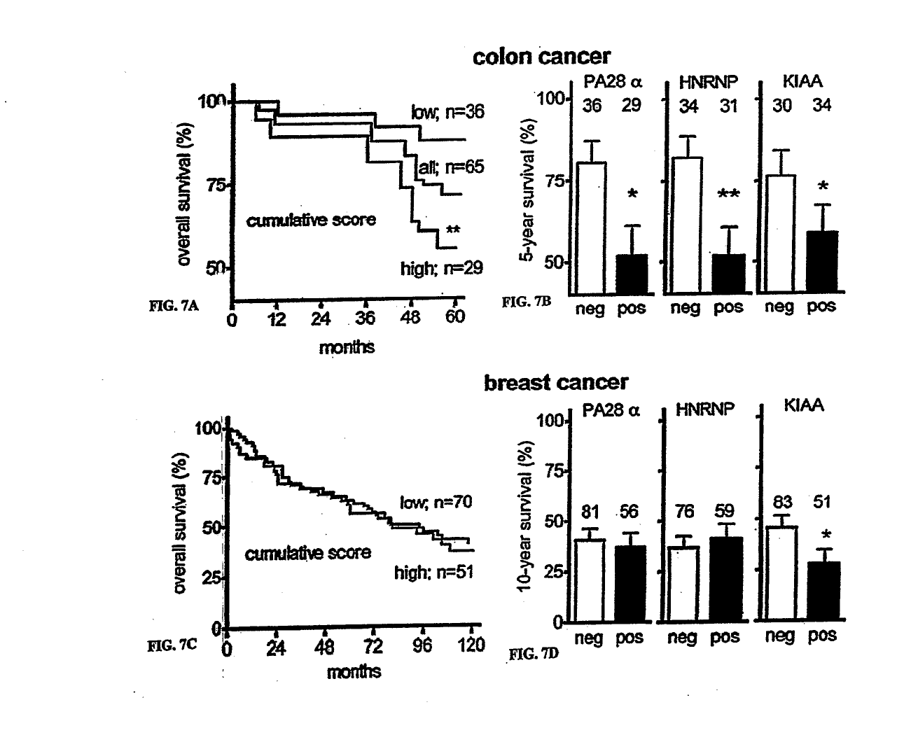

Also provided herein are compositions and methods for determining survival rates (e.g., the probability of survival) in an individual diagnosed with colon cancer, whether or not the cancer has metastasized to a secondary tissue source, such as for example, the liver, comprising detecting or measuring the presence of and/or the presence of elevated levels of one or more of 1) a polynucleotide disclosed herein in FIGS. 2A-2C, or a complement thereof; 2) a polynucleotide encoding any one of the polypeptides disclosed herein in FIGS. 3A-3E, or a complement thereof; 3) a polynucleotide encoding any one of the polypeptides disclosed herein in FIGS. 10 through 13, or a complement thereof; 4) a polypeptide disclosed herein in FIGS. 3A-3E, and/or a fragment thereof; or 5) a polypeptide disclosed herein in FIGS. 10-13, or fragment thereof, wherein the presence of or presence of elevated levels of anyone of 1) through 5) as compared to a control, is correlated with a decrease in survival rates. Provided herein are methods for determining survival rates in an individual diagnosed with colon cancer, whether or not the cancer has metastasized to a secondary tissue source, such as for example, the liver, comprising measuring the presence of and/or the presence of elevated levels of one or more of PA28alpha, HNRNPA1, and KIAA1407, or polynucleotides encoding them. Also provided herein are methods for determining survival rates in an individual diagnosed with colon cancer, whether or not the cancer has metastasized to a secondary tissue source, such as for example, the liver, comprising measuring the presence of and/or the presence of elevated levels of any one, two or three of PA28alpha, HNRNPA1, and KIAA1407, or polynucleotides encoding them, in a sample from the individual. In some examples, the survival rates are five year survival rates and in other examples are ten year survival rates. Such methods comprise measuring any one, two or three of PA28alpha, HNRNPA1, and KIAA1407, or polynucleotides encoding them, alone or together with any one or more of the other polypeptides disclosed herein in FIGS. 3A-3E, or polynucleotides encoding them, such as those disclosed herein in FIGS. 2A-2C, or the polypeptides disclosed in FIGS. 10-13, or the polynucleotides encoding them. The measurements of any two of or three of PA28alpha, HNRNPA1, and KIAA1407 may be considered individually as compared to an appropriate control or considered cumulatively, that is, in an additive manner. For example, for cumulative measurements of PA28alpha, HNRNPA1, and KIAA1407, measurement of each marker is given a value from between 1 and 3, with 1 being a low measurement value and 3 being a high measurement value; then the measurements are added. As shown in FIG. 4D, a cumulative measurement of PA28alpha, HNRNPA1, and KIAA1407 of >0 has the highest survival rates, whereas a cumulative measurement of PA28alpha, HNRNPA1, and KIAA1407 of >8 has the lowest survival rate. FIGS. 6A-6B show the correlation of PA28alpha, HNRNP and KIAA, cumulatively (FIG. 6A) and individually (FIG. 6B) with 5 year (60 months) survival rates in individuals diagnosed with colon cancer. As shown in FIG. 6A, the samples from individuals showing the lowest cumulative levels of PA28alpha, HNRNP and KIAA correlated with the highest 5 year survival rates whereas the samples from individuals showing the highest cumulative levels of PA28alpha, HNRNP and KIAA correlated with the lowest 5 year survival rates. As shown in FIG. 6B, the presence of PA28alpha alone, HNRNP alone or KIAA alone in samples from individuals is correlated with reduced 5 year survival as compared to samples that are negative for any of PA28alpha, HNRNP, or KIAA. FIGS. 7A-7D demonstrate that this correlation does not apply to samples from individuals subject to breast cancer. The lower the cumulative levels of PA28alpha, HNRNP and KIAA the greater the probability of not having metastasis while the higher the cumulative levels of PA28alpha, HNRNP and KIAA, the greater the probability of having metastasis.

In another example, occult micrometastasis could be identified using PA28alpha, HNRNPA1, and KIAA1407 in imaging techniques and then treated at the time of surgery even when no metastatic disease has been otherwise identified using conventional techniques.

As will be appreciated by one of skill in the art, appropriate controls may be used in the methods disclosed herein. Appropriate controls include, but are not limited to a population control, non-cancer samples; non-colon cancer samples for which PA28alpha, HNRNPA1, and KIAA1407 do not selectively target, such as for example, breast cancer; or baseline values for a population or an individual, such as a baseline sample from an individual taken at an earlier time. In some examples, both positive and negative controls are used, or non-metastatic control samples are used.

In some examples, the samples are biopsy samples from an individual at risk for colon cancer; subject to colon cancer, wherein the cancer can be any stage of cancer including Stage I, H, III or IV; at risk for or subject to recurrence of primary colon cancer; at risk for or subject to metastasis, whether or not the primary colon cancer has been surgically excised; and in some examples, the samples are collected at the time of surgical excision of the primary colon cancer. Staging systems for colorectal cancer are disclosed in for example, Robbins Textbook of Pathology and Meyerhardt et al., (2005, New England Journal of Medicine, 352:4).

Methods for measuring the presence of targeting domains disclosed herein, such as for example, PA28alpha, HNRNPA1, and KIAA1407, or the presence of elevated levels of PA28alpha, HNRNPA1, and/or KIAA1407, or polynucleotides that encode them, are known in the art and described herein. In some examples, polynucleotides encoding PA28alpha, HNRNPA1 or KIAA1407, or PA28alpha, HNRNPA1, and KIAA1407 are used alone; together, such as in a set; or together with polynucleotides that encode other liver targeted domains as described herein in the Figures; to hybridize tissue samples, such as biopsy samples, in situ, such as for example by methods described herein in the examples. In other examples, immunohistochemistry methods such as those described herein and known in the art are used to detect the presence of targeting domains on samples, such as for example, biopsy samples, by using antibodies specific for the targeting domains. Accordingly, the present invention also provides kits that comprise polynucleotides that encode PA28alpha, HNRNPA1, and/or KIAA1407, and/or reagents specific for PA28alpha, HNRNPA1, and/or KIAA1407, such as antibodies or fragment thereof, that might be used in the methods as described herein. In some examples, a kit for use in the determination of survival rates, such as for example, five and ten year survival rates, comprises polynucleotides that encode PA28alpha, HNRNPA1, and/or KIAA1407; and/or reagents specific for PA28alpha, HNRNPA1, and/or KIAA1407, such as antibodies or fragments thereof, along with instructions for their use.

Method of Identifying Targeting Domains

The ability of tumors to metastasize is, at least in part, the result of genes whose products comprise a targeting domain which selectively directs a tumor cell from the primary tumor to secondary tissue metastatic sites. By way of example, the targeting domain may act as a receptor to a ligand on the surface of vessels in the specific target organs or as ligands to receptor proteins on the surface of vessels in those organs. Methods are disclosed herein which were used to identify polynucleotides associated with metastasis and the targeting domain(s) encoded by such polynucleotides. These polynucleotides and/or polypeptides may provide an understanding of the mechanism of action of metastatic tumors at a molecular level.

The present invention discloses the preparation of libraries created from primary tumors that can be utilized for the identification of homing genes. Primary tumors that are known to metastasize to particular organs are selected for the production of a phage library. That library is then injected into an appropriate model. The model is preferably a mammal such as, for example, a human, a mouse or a rabbit, but may also be any other mammal. Alternatively, the method may utilize any animal, including non-mammals, that allows for the injection of phage library and demonstrates a significant response.

Human tumor libraries are often not available or may not yield the greatest response in a non-human model. Accordingly, the present invention comprises tumor libraries derived from animals that show a response in the model.

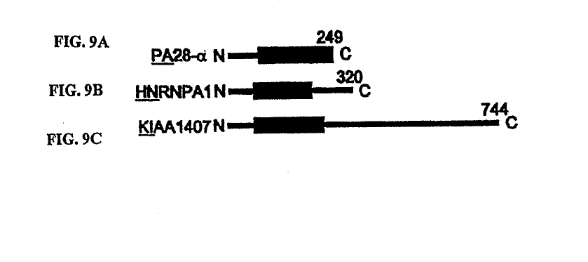

Accordingly, disclosed herein are polynucleotides encoding a targeting domain associated with metastasis of tumor cells, that were identified by the methods as disclosed herein including the method comprising: (a) administering a phage displaying libraries comprising a collection of phages containing polynucleotides from a primary tumor cell into a subject; (b) selecting phage that localize in a target organ or tissue; (c) collecting phage from the selected organ or tissue; (d) repeating steps (a) and (c) for one or more cycles; and (e) identifying one or more polynucleotides encoding a targeting domain or fragment thereof from selected phage that are associated with tumor cell metastasis. Such polynucleotides include the polynucleotides disclosed herein in FIG. 2A-2C; polynucleotides that encode the polypeptides of FIG. 3A-3C; fragments thereof, and complements thereof. In some examples the polynucleotides encode PA28alpha, HNRNPA1, and KIAA1407, and in other examples, comprise or consist essentially of the polypeptides for PA28alpha, HNRNPA1, and KIAA1407, or fragments thereof, such as the targeting domains described in FIG. 9.

Any library may be used in the method described herein. Standard methods may be utilized to create the library or the library may be obtained from a commercial source. Examples of libraries that may be used in the method include, but are not limited to libraries created from primary tumors of lung, stomach, colon, rectum, prostate, pancreas, liver, leukemia, breast, uterus, ovary, melanoma, urinary tract, bladder, cervix, lymph, brain, nervous system or combinations thereof, peptide libraries, or libraries comprising molecules sharing common functional domains or sequence (e.g., kinases, cytokines, growth factors etc) or polynucleotides from any eukaryarotic cell. In one embodiment, a library used in the method is reused with the clones isolated from the first screen subtracted out from the library to minimize repetitive isolation of the same clone. Methods of creating subtraction libraries are well known in the art.

Any phage may be used to create the library. Preferably, the phage used in the creation of the library has one or more of the following characteristics: the ability to contain and relatively large polynucleotides, such as, for example, between about 300-3000 nucleotides and/or expresses the clone from the library at a low copy number, such as, for example, between about 0.1 copy to about 1 copy per phage. Such phages are commercially available (e.g., a T7Select vector using T7Select 1-1phage). By way of example, a phage display library may comprise and express polynucleotides isolated from a primary tumor, such as, for example, colon cancer or from a cell line such as, for example, a colon cancer cell line (e.g., LSI74T; American tissue culture collection, ATCC, Rockville, Md.). Preferably, the phage themselves (i.e. phage without a recombinant insert) have a low relative retention to target organs or cells. Retention, which may relate to direct binding, non-specific association, or active uptake, will cause phage to nonspecifically associate with target cells. By identifying and selecting only phage with low retentions by target cells, the highest selectivity can be achieved.

Relative retention of phage to target tissue is preferably less than 50%, more preferably less than 10%, and still more preferably less than 1%.

The library is administered to any subject, preferably a mouse or other mammal. The animal may be a normal animal or an animal model of disease. Alternatively, the library may be contacted with in vitro systems or models. In an animal, such as for example, a mouse, a volume of between about 10 microliters to about 100 microliters containing between about 10⁷ to about 10¹⁰ phage is administered to a mouse. Phage, based on the expression product displayed, target to selected organs, tissues or other areas of the body. Accordingly, the library is administered and allowed to circulate for a time to sufficient to allow binding to the target tissue and/or organ of the binding domains expressed in the library. The optimal circulation time will vary with the size/weight of the animal, volume and/or complexity of the library. By way of example, for a mouse circulation time may be preferably between about one minute to about ten minutes.

After sufficient circulation time the animal is euthanized and the target organs collected for analysis. The method described herein may be further enhanced by further comprising perfusing the anesthetized animal with an isotonic salt solution with or without proteins (e.g., BSA) to minimize non-specific binding of phage. Examples of isotonic salt solutions include, but are not limited to phosphate buffer. Perfusion is continued, preferably until desanguination (e.g., little or no blood exits the vena cava, organs appear white in color.) By way of example, volumes of between about 1 to about 100, preferably about 20 times the volume of the animal may be used.

Any organ or tissue may be harvested for analysis. By way of example, these may include, but are not limited to bone marrow, lung, skin, liver and/or brain. Generally the tissue or organ harvested will be selected based on the origin of the library. By way of example, metastasis in colon cancer is often to the liver, marrow, lung and/or bone marrow. If the library used in the method comprises polynucleotides from a primary colon cancer tumor or cell line, liver lung and/or bone marrow can be harvested. Phage are collected from the selected tissues and/or organs, amplified, if necessary, and injected into another animal. Through successive rounds of injection, selection, and amplification, a collection of phage can be isolated that are specific for the selection criteria. By way of example, between about two to about five rounds of injection, selection, and amplification may performed. These collections can be further selected or the polynucleotides from individual or groups of phage isolated and identified. Polynucleotides identified by these methods can be used for both diagnostic and therapeutic purposes. The polynucleotide expression products identified may be useful to distinguish metastatic from non-metastatic disease. Alternatively, the products may be useful in identifying new therapies for the treatment of metastatic and for the screening of promising pharmaceutical products.

The method described herein for identifying targeting domains may also be utilized to identify targeting domains in other diseases or disorders. By way of example, such diseases or disorders may include, but are not limited to, arteriosclerosis, coronary artery disease, stroke, diabetic vascular damage (e.g., kidney vascular damage) or retinopathy. Examples of animals models to be used in the methods described herein include, but are not limited to, cardiovascular diseases in pig, rat, rabbit arterial stenosis and vascularization. (e.g., Goodman and Gilman's: The Pharmaceutical Basis of Therapeutics Pergamon Press (1990)).

Polynucleotides

Provided herein are polynucleotides identified by the method described herein. Such polynucleotides include the polynucleotides disclosed herein in FIG. 2A-2C; polynucleotides that encode the polypeptides of FIG. 3A-3C; polynucleotides that encode the polypeptides of FIG. 10-13, fragments thereof, and complements thereof. In some examples the polynucleotides encode PA28alpha, HNRNPA1, and/or KIAA1407, or fragments thereof and in other examples, comprise or consist essentially of polynucleotides that encode targeting domains of PA28alpha, HNRNPA1, and/or KIAA1407 as described herein. The term polynucleotide is used broadly and refers to polymeric nucleotides of any length (e.g., oligonucleotides, genes, small inhibiting RNA etc). The polynucleotide of the invention may be, for example, linear, circular, supercoiled, single stranded, double stranded or branched. It is, however, understood by one skilled in the art that due to the degeneracy of the genetic code variations in the polynucleotide sequences shown will still result in a polynucleotide sequence capable of encoding a targeting domain as disclosed herein in FIGS. 3A-3E. Such polynucleotide sequences are therefore functionally equivalent to the sequence set forth in FIG. 2A-2C and are intended to be encompassed within the present invention. Further, a person of skill in the art will understand that there are naturally occurring allelic variations of the polynucleotide sequences shown herein these variations are also intended to be encompassed by the present invention.

Provided herein are methods that comprise the use of polynucleotides that comprises the polynucleotide sequence encoding PA28alpha, HNRNPA1, and KIAA1407, or a targeting domain thereto. In yet other examples, provided herein are methods that comprise the use of the isolated polynucleotides that encode PA28alpha, HNRNPA1, and/or KIAA1407 either alone or together with polynucleotides that encode other targeting domains as described herein.

Polynucleotides that hybridize under stringent conditions to a polynucleotide comprising the sequence as shown in SEQ ID NOS. 1, 2, 3, 4, 5, 6, 7, 8, or 9 can also be used in the methods disclosed herein. Hybridization reactions can be performed under conditions of different “stringency”. Conditions that increase stringency of a hybridization reaction are widely known and published in the art. See, for example, Sambrook et al. (1989). Examples of relevant conditions include (in order of increasing stringency): incubation temperatures of 25° C., 37° C., 50° C. and 68° C.; buffer concentrations of IO×SSC, 6×SSC, 4×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. In a preferred embodiment hybridization and wash conditions are done at high stringency. By way of example hybridization may be performed at 50% formamide and 4×SSC followed by washes of 2×SSC/formamide at 50° C. and with 1×SSC (see example).

Polypeptides

The term polypeptide is used broadly herein to include peptide or protein or fragments thereof. Also intended to be encompassed are peptidomimetics, which include chemically modified peptides, peptide-like molecules containing nonnaturally occurring amino acids, peptoids and the like, have the selective binding of the targeting domains provided herein. (“Burger's Medicinal Chemistry and Drug Discovery” 5th ed., vols. Ito III (ed. M. E. Wolff; Wiley Interscience 1995).

In some examples of the methods, the polypeptide comprises the amino acid sequence of PA28alpha, HNRNPA1, or KIAA1407. This invention further includes polypeptides or analogs thereof having substantially the same function as the polypeptides of this invention. Such polypeptides include, but are not limited to, a substitution, addition or deletion mutant of the polypeptide. This invention also encompasses proteins or peptides that are substantially homologous to the polypeptides.

In specific embodiments, the present invention provides a homing domain of a liver targeting polypeptide. For example, such homing domain of a liver targeting polypeptide is selected from: a C-terminal portion of PA28alpha (e.g., amino acids 98 to 249 of GenBank Accession No. AAV38144), a central portion of HNRNP A1 (e.g., amino acid residues 38 to 189 of GenBank Accession NP_{—}112420), and an N-terminal region in a predicted protein of no known function, KIAA1407 (e.g., amino acids 96 to 293 of GenBank Accession No. BAA92645).

The term “analog” includes any polypeptide having an amino acid residue sequence substantially identical to at least one the polypeptide sequences specifically shown herein (e.g., FIG. 3A-3E) in which one or more residues have been conservatively substituted with a functionally similar residue and which displays the functional aspects of the polypeptides as described herein.

Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The phrase “conservative substitution” also includes the use of a chemically derivatized residue in place of a non-derivatized residue. “Chemical derivative” refers to a subject polypeptide having one or more residues chemically derivatized by reaction of a functional side group. Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those proteins or peptides which contain one or more naturally-occurring amino acid derivatives of the twenty standard amino acids.

For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides of the present invention also include any polypeptide having one or more additions and/or deletions or residues relative to the sequence of a any one of the polypeptides whose sequences is described herein.

Methods of Prognosing and/or Diagnosing

Provided herein are methods for determining the presence of PA28alpha, HNRNPA1, and/or KIAA1407, or polynucleotides encoding them, in a sample from an individual at risk for or subject to cancer, such as colon cancer, wherein the presence of or presence of elevated levels of PA28alpha, HNRNPA1, and/or KIAA1407, or polynucleotides encoding them, is correlated with increased risk of metastasis, such as metastasis to the liver. In some examples, the presence of cumulative elevated levels of PA28alpha, HNRNPA1, and KIAA1407, or polynucleotides encoding them (relative to a control, such as a non-colon cancer sample, or a non-cancer sample) are correlated with an increased risk of metastasis.

Also provided herein are methods for determining the risk of metastasis in an individual diagnosed with cancer, such as for example, colon cancer; comprising detecting the presence of and/or measuring the levels of PA28alpha, HNRNPA1, and/or KIAA1407, or polynucleotides encoding them, in a sample from the individual, wherein the presence and/or presence of elevated levels of PA28alpha, HNRNPA1, and/or KIAA1407, or polynucleotides encoding them (relative to a control, such as a non-colon cancer sample, or a non-cancer sample) is correlated with an increased risk of metastasis.

Also provided herein are methods for determining survival rates in an individual diagnosed with colon cancer, whether or not the cancer has metastasized to a secondary tissue source, such as for example, the liver, comprising measuring the presence of and/or the presence of elevated levels of any one, two or three of PA28alpha, HNRNPA1, and KIAA1407, or polynucleotides encoding them (relative to a control, such as a non-colon cancer sample, or a non-cancer sample), in a sample from the individual wherein the presence of or the presence of elevated levels of any one, two or three of PA28alpha, HNRNPA1, and KIAA1407, or polynucleotides encoding them, is correlated with a decrease in survival rates. In some examples, the presence of elevated cumulative levels of PA28alpha, HNRNPA1, and KIAA1407, that is, the additive measurement of PA28alpha, HNRNPA1, and KIAA1407 together, is correlated with a decrease in survival rates (that is, correlated with an increase in mortality rates), and in some examples, is correlated with a decrease in five year and ten year survival rates. Such methods comprise measuring any one, two or three of PA28alpha, HNRNPA1, and/or KIAA1407, or polynucleotides encoding them, alone or together with any one or more of the other liver homing domains disclosed herein in FIGS. 3A-3E, or polynucleotides encoding them, such as the polynucleotides disclosed in FIGS. 2A-2C. In some examples, the samples are biopsy samples.

The level of metastasis may be correlated to the level of primary tumor growth leading to increased neoplastic embolism which then increases the level of neoplastic aggregates in the blood stream. A method of quantitatively measuring the RNA transcription product in the blood would allow an estimation of primary tumor growth and the metastatic potential of the tumor. In some cases, quantitative measurements can be made with a PCR or, alternatively, other methods to quantitatively measure transcription may be desirable. In many situations, different primary tumors metastasize to different organs.

The methods provided herein maybe prognostic (e.g., detect subclinical metastasis, detection of subclinical metastasis in at risk patients, risk of metastasis) or diagnostic (e.g., detect metastasis, monitor disease progression or treatment). One embodiment provides methods of prognosing and/or diagnosing metastatic disease in a subject. In one embodiment, the method comprises detecting the level of a polynucleotide: encoding a polypeptide comprising the targeting domain in a sample obtained from a subject, wherein a higher level of the polynucleotide relative to a control sample (e.g., population controls or nonmetastatic control sample) is indicative of metastatic disease. In another embodiment, the method comprises detecting the presence or absence of a polynucleotide encoding a polypeptide comprising the targeting domain in a sample obtained from the subject, wherein the presence of the polynucleotide is indicative of metastatic disease. Conventional methodology may be used to detect the polynucleotides in the method described herein. Examples include, but are not limited to, PCR analysis, RT-PCR, Northern analysis or microarrays as described herein below. Examples of a sample obtained from a subject include, but is not limited to, blood, biopsy sample, pathology sample, urine or cerebrospinal fluid.

Yet another aspect of this invention provides methods of prognosing, imaging and/or diagnosing metastatic disease in a subject. In one embodiment, the method comprises detecting the level of a polypeptide comprising the targeting domain in a sample obtained from a subject, wherein a higher level of the polypeptide relative to a control sample (e.g., population controls or nonmetastatic control sample) is indicative of metastatic disease. In another embodiment, the method comprises detecting the presence or absence of a polypeptide comprising a targeting domain in a sample obtained from the subject, wherein the presence of the polypeptide is indicative of metastatic disease. Conventional methodology may be used to detect the polypeptides in the method described herein.

Examples include, but are not limited to, Western blot analysis or protein microarrays. Other methods of quantitative analysis of proteins include, for example, proteomics technologies such as isotope coded affinity tag reagents, MALDI TOF/TOF tandem mass spectrometry, and 2D-gel/mass spectrometry technologies. These technologies are commercially available from, for example, Large Scale Proteomics, Inc. (Germantown, Md.) and Oxford Glycosystems (Oxford UK). Methods for quantitatively measuring proteins such as ELISA analyses are well known. Kits for measuring levels of many proteins using ELISA assays are commercially available from many suppliers. In addition, methods for developing ELISA assays in the laboratory are well known. See for example Antibodies: A Laboratory Manual (Harlow and Lane Eds. Cold Spring Harbor Press). Antibodies for use in such ELISA methods either are commercially available or are prepared using well-known methods. Examples of a sample obtained from a subject include, but is not limited to, blood, biopsy sample, pathology sample, urine or cerebrospinal fluid.

Microarrays

In yet another aspect of the invention, provided herein are microarrays comprising polynucleotides that encode PA28alpha, HNRNPA1, or KIAA1407, along with appropriate controls, such as a positive and negative control or the controls as described herein. In some examples, polynucleotide sequences include those shown in FIGS. 2A-2C, as well as their complements. In some examples, polypeptide sequences include those shown in FIG. 3A-3E and those shown in FIGS. 10-13. Methods of making microarrays are known in the art. By way of example, one or more of the polynucleotide sequences described herein may comprise an array of polynucleotides attached to a support (e.g., dot blots on a nylon hybridization membrane, Sambrook et al., or Ausubel et al) that is contacted with the nucleic acids isolated from, for example, a patient sample.

Microarrays may be a solid phase on the surface of which are immobilized a population of the nucleic acids of the invention. Microarrays can be generated in a number of ways. The polynucleotides can be attached to a solid support or surface, which may be made from, for example, glass, plastic (e.g., polypropylene, nylon), polyacrylamide, nitrocellulose, or other materials.

Methods for attaching the nucleic acids to the surface of the solid phase include, but are not limited to, printing on glass plates (Schena et al, 1995, Science 270:467-470; DeRisi et al, 1996, Nature Genetics 14:457-460; Shalon et al., 1996, Genome Res. 6:639-645; and Schena et al., 1995, Proc. Natl. Acad. Sci. U.S.A. 93:10539-11286); or ink jet printer.

The microarrays can also be high-density oligonucleotide arrays. Techniques are known for producing arrays containing thousands of oligonucleotides complementary to defined sequences (see, Fodor et al., (1991) Science 251:767-773; Pease et al., (1994) Proc. Natl. Acad. Sci. U.S.A. 91:5022-5026; Lockhart et al., (1996) Nature Biotechnology 14:1675; U.S. Pat. Nos. 5,578,832; 5,556,752; and 5,510,270; Blanchard et al., Biosensors & Bioelectronics 11:687-690). Other methods for making microarrays may also be utilized (Maskos and Southern, (1992) Nuc. Acids. Res. 20:1679-1684; U.S. Pat. No. 6,136,592; WO 200054883; WO 200055363; WO 200053812; WO 200014273). The microarrays may be used as is or incorporated into a biochip, multiwell or other device.

Antibodies

The invention also provides antibodies, or fragments thereof which specifically bind PA28alpha, HNRNPA1, and/or KIAA1407 for use in the methods disclosed herein, or which specifically bind one or more of the polypeptides disclosed herein in the FIGS. 3A-3E. In some examples, provided herein are a set of antibodies, or fragments thereof, specific for PA28alpha, HNRNPA1, and/or KIAA1407. The antibodies can be monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv) antibodies, mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies). The epitope(s) can be continuous or discontinuous. The antibodies may be made by any method known in the art and tested by the method described herein. In an alternative, antibodies may be made recombinantly and expressed using any method known in the art. In another alternative, antibodies may be made recombinantly by phage display technology. See, for example, U.S. Pat. Nos. 5,565,332; 5,580,717; 5,733,743; 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors.

Yet another aspect of the invention relates to detection of metastatic disease in a subject, such as a human utilizing one or more antibodies described herein, such as antibodies to PA28alpha, HNRNPA1, and/or KIAA1407, or fragments thereof, or epitopes thereof coupled to a radiologic (e.g., I¹²⁵) or other imaging molecules (e.g., dyes, pigments or fluorescent molecules such as luciferase, fluoroscein or commercially available fluorescent molecules from quantum.com). The antibodies may be coupled to the radiologic or imaging molecule by methods known in the art.

Another embodiment relates to the polypeptides comprising the targeting domains described herein, such as for example, the targeting domains for PA28alpha, HNRNPA1, and/or KIAA1407, shown in the Figures and/or as identified by phage display, coupled to a moiety, such as a therapeutic moiety or a detection moiety. The moiety may be any molecule.

Examples of therapeutic moieties include, but are not limited to, ricin, radioisotopes, clotting agents, thrombolytic factors, chemotherapeutic and radiosensitizing agents, anti-angiogenesis agents, anti-motility agents, and immunomodulatory agents. Examples of a detection moiety include, but are not limited to, radioisotopes, dyes, pigments, or fluorescent molecules such as luciferase, fluoroscein or commercially available fluorescent molecules from quantum-com. The polypeptide may be coupled to the radiologic or imaging molecule by methods known in the art and used to target delivery of the therapeutic or detection moiety to the liver.

Screening Methods

Provided herein are methods for screening for a candidate agent that blocks, suppresses or reduces (including significantly) the binding of the targeting domains, in particular the binding of PA28alpha, HNRNPA1, and/or KIAA1407 to tissues. Exemplary types of agents that may be screened for ability to inhibit one or more of the targeting domains described herein include, but are not limited to, an antibody, an anti-sense molecule directed to one or more polynucleotide sequences encoding the targeting domain, an NGF inhibitory compound, a structural analog, a dominant-negative mutation, immunoadhesin, small molecules having a molecular weight of 100 to 20,000 daltons, 500 to 15,000 daltons, or 1000 to 10,000 daltons. Libraries of small molecules are commercially available.

Design of Methods of Treatment

The diagnostic and prognostic methods as described herein, as well as methods that predict or identify survival rates, such as for example, five and ten year survival rates, can be used to design appropriate therapeutic intervention. For example, for an individual with early stage cancer, such as colon cancer, that has not metastasized, methods for detecting the presence of elevated cumulative levels of PA28alpha, HNRNPA1, and/or KIAA1407, or polynucleotides encoding them, can help a physician determine if frequent diagnostic assessment is necessary or if aggressive therapeutic intervention is necessary, or if it is necessary to treat metastasis in the individual. A physician will be able to determine appropriate therapeutic intervention based on the methods disclosed herein and conventional methods known in the art.

In some examples, the method comprises administering to a subject in need of such treatment a targeting domain linked to a therapeutic agent in an amount effective to treat the metastasis, or an effective amount of a composition that inhibits the metastasis (e.g., collection of phage or phage expression products identified by the method herein; a targeting domain linked to a therapeutic agent and/or an antibody directed against a polypeptide comprising a targeting domain).

In other examples, provided herein are kits and compositions comprising polynucleotides encoding one or more of PA28alpha, HNRNPA1, and KIAA1407 polypeptides, or reagents specific thereto, such as antibodies, specific for couple moieties described herein.

Methods of Inhibiting Gene Expression

The identification of genes that allow primary tumors to specifically target distant organs as sites of secondary metastasis provides new therapeutic methods of treatment. For example, the ability of tumor cells to induce transcription of an identified gene may be altered. Methods for down regulating genes are well known. It has been shown that antisense RNA introduced into a cell will bind to a complementary mRNA and thus inhibit the translation of that molecule. In a similar manner, antisense single stranded cDNA may be introduced into a cell with the same result. Further, co-suppression of genes by homologous transgenes may be effected because the ectopically integrated sequences impair the expression of the endogenous genes (Cogoni et al. Antonie van Leeuwenhoek, 1994; 65(3):205-9), and may also result in the transcription of antisense RNA (Hamada, W. and Spanu, P D; Mol. Gen. Genet. 1998). Methods of using short interfering RNA (RNAi) to specifically inhibit gene expression in eukaryotic cells have recently been described. See Tuschl et al., Nature 411:494-498 (2001). In all of the above methods, transfection of cells can be effected using adeno-viral or other viral vectors. In addition, stable triple-helical structures can be formed by bonding of oligodeoxyribonucleotides (ODNs) to polypurine tracts of double stranded DNA. (See, for example, Rininsland, Proc. Nat'l Acad. Sci. USA 94:5854-5859 (1997). Triplex formation can inhibit DNA replication by inhibition of transcription of elongation and is a very stable molecule. Accordingly, provided herein are methods for inhibiting expression of PA28alpha, HNRNPA1, and/or KIAA1407.

Methods to Inhibit the Activity of Specific Proteins

The present invention provides methods of inhibiting the activity of PA28alpha, HNRNPA1, and/or KIAA1407 at secondary metastatic sites. Specifically, the site of metastasis may be targeted due to a surface protein found in the vascular walls of the endothelium at that site. It will then be possible to affect the expression of that gene down regulating it such that the metastatic tumor cells are not able to enter the endothelium and consequently will be unable to promote secondary tumor growth.

When a specific protein has been implicated in the metastatic ability of primary tumor cells its activity can be altered by several methods. First, specific antibodies may be used to bind the target protein thereby blocking its ability to attract secondary metastasis. In addition, antibodies against the homing protein may be used with a similar result. Such antibodies may be used to bind the protein thereby blocking its activity. Specific antibodies may be obtained though the use of conventional hybridoma technology or may be isolated from libraries commercially available from Dyax (Cambridge, Mass.), MorphoSys (Martinsried, Germany), Biosite (San Diego, Calif.) and Cambridge Antibody Technology (Cambridge, UK). In addition, identified proteins may act as cellular receptors.

Identification of such receptors will allow the design of specific ligand antagonists which may affect the metastasis by either 1) binding to the receptor on the metastasizing tumor cell or 2) binding to the target of the metastatic cell in the vasculature at the site of the secondary tumor.

In addition, identification of metastatic proteins also allow for the design of drugs to specifically target both the primary tumor and the secondary tumor. For example, a protein on the surface of a tumor cell that allows it to home-in at a site of secondary metastasis will also allow the design of drags that bind to that protein at the site of the primary tumor, as well as, to tumor cells which are embolized in the blood. Similarly, identification of such proteins will allow the design of a drug or agent having an epitope similar to the identified gene product allowing the drug to home-in at the site of the metastases. Thus specific targeting of the primary and secondary tumors may be effected.

In addition, the present invention provides kits comprising primers specific for polynucleotides encoding PA28alpha, HNRNPA1, and KIAA1407, including kits comprising primers specific for PA28alpha, HNRNPA1, and/or KIAA1407, along with appropriate controls and instructions for use. Because metastatic tumor cells travel in the blood stream, use of such kits will only necessitate the drawing of blood from a patient and the use of PCR to perform RT-PCR to identify clinically the presence of a tumor, as well as, its metastatic potential.

The following examples illustrate embodiments of the invention, but should not be viewed as limiting the scope of the invention.

### EXAMPLES

### Example 1

### In Vivo Phage Display Identification of Metastatic Cancer Genes

Materials and Methods

Tumor cell lines: Colon cancer cell line (LS174T; American tissue culture collection, ATCC, Rockville, Md.), Melanoma cell line (1205LU; a gift from M. Heerlyn, Wistar Institute, Philadelphia).

Animal: Athymic nude mice were used for the in vivo selection studies.

To identify proteins that can guide organ specific tumor metastasis, a model human colon cancer cell line, LS174T, which will metastasize to the liver in athymic nude mice when implanted into the colon was used. Without being bound by theory, it was hypothesized that phage displaying fragments of liver homing proteins on their surface would be retained by the liver vascular bed when injected into the vena portae, i.e. the route of tumor cell trafficking from the colon to the liver. Several rounds of in vivo selection would then enrich for liver homing genes expressed in the metastatic cell line. To generate a library that presents putative organ homing domains as a capsid fusion protein on the surface of T7 phage, size-selected (>200 bp), random-primed cDNAs from mRNA of LS174T cells were inserted into the 10B gene of T7 phage. PCR amplification of the cDNA inserts using the whole library as well as individually picked plaques showed a size range of 300 to 2,000 by and the phage are designed to express on average only one copy of the respective fusion protein per phage to accommodate fusion proteins up to 3,000 amino acids.

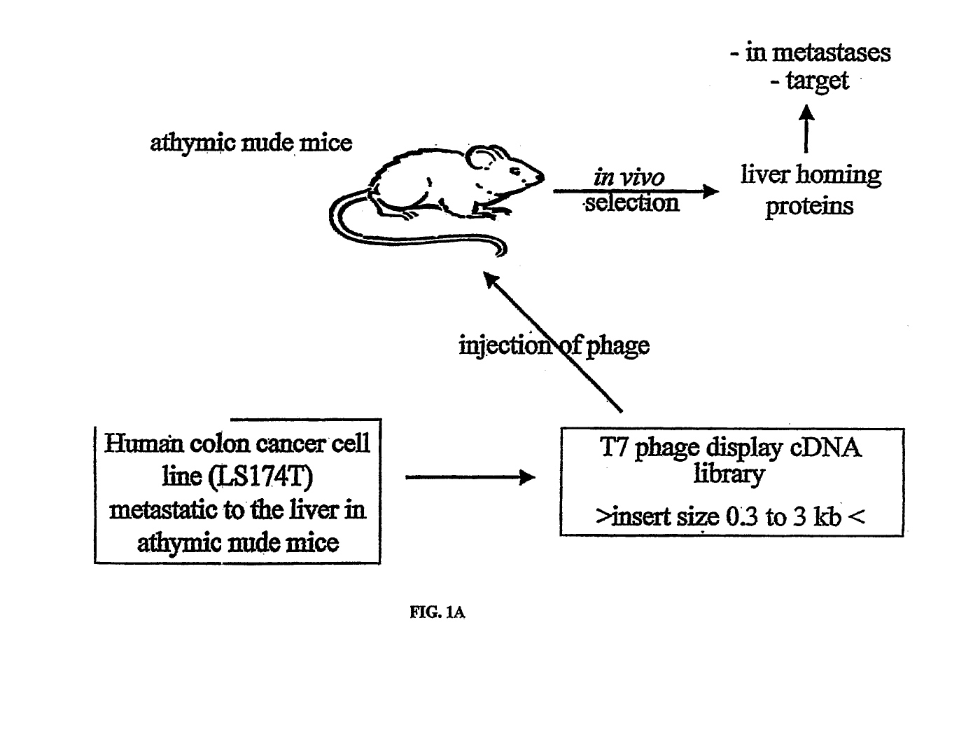

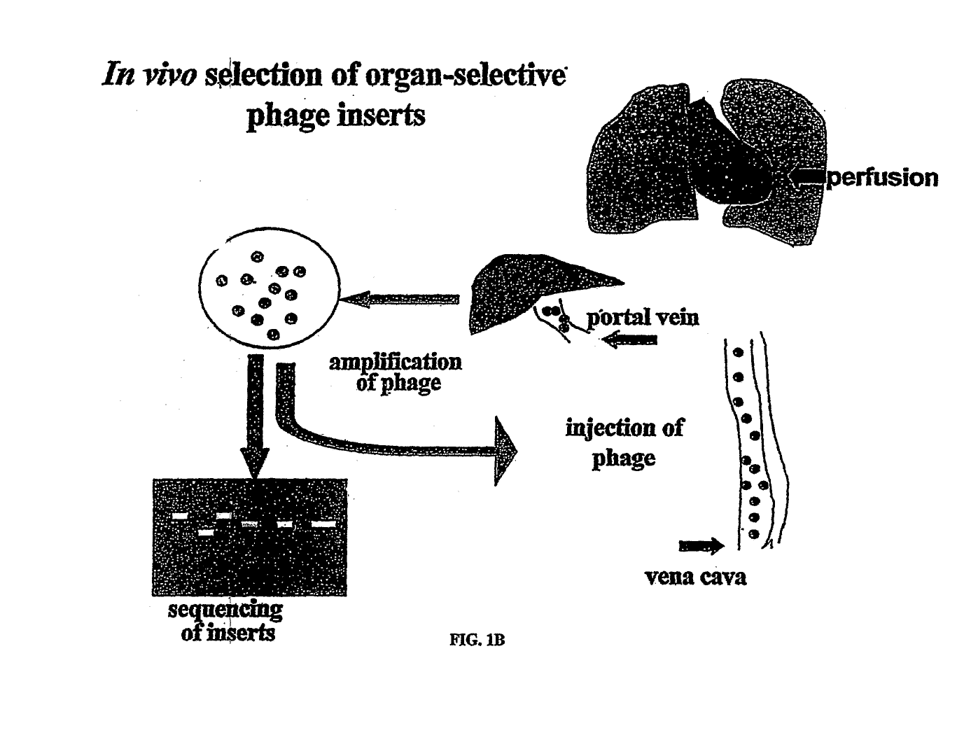

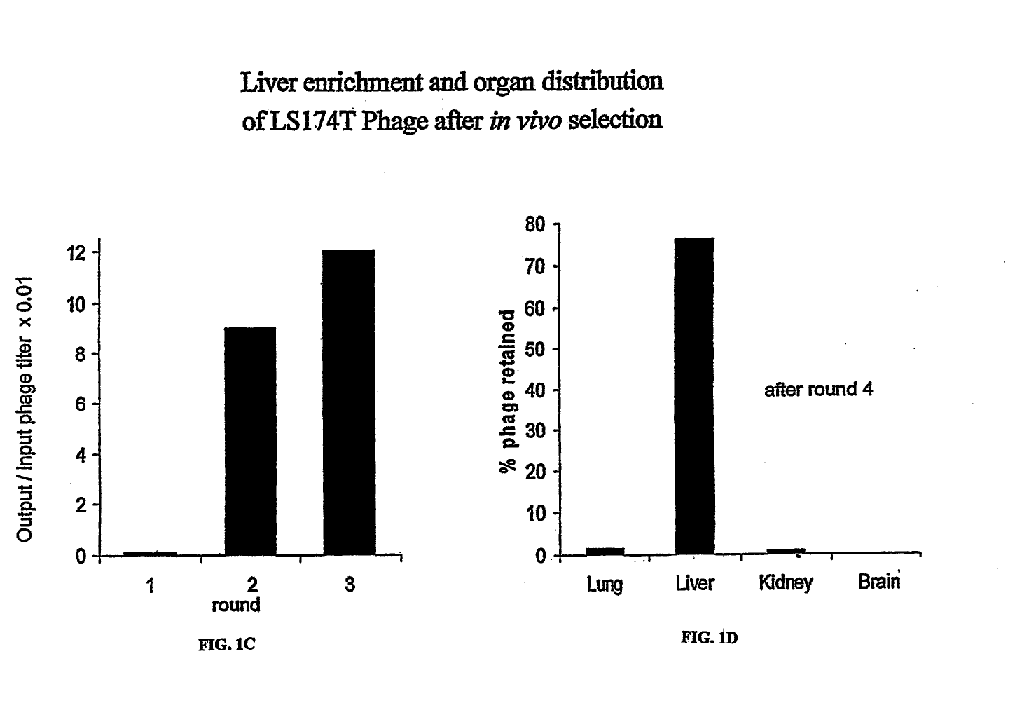

Generation of cDNA phage libraries: cDNA libraries of the cancer cell lines LS174T and 1205LU were constructed by using the OrientExpress directional random primer strategy (Novagen, Inc.; Darmstadt, DRG). The cDNA were then inserted into a T7Select vector using T7Select 1-1 (up to 1200 amino acids and approximately 1 copy/phage). The cDNA was inserted into the gene of capsid protein 10 and the fusion protein expressed on the surface of the phage capsid (FIG. 1). Using 300 nucleotides as the minimum size of cDNA to be inserted into each phage, a library having a diversity of 1-4×10⁶ was obtained.

Once the vectors, which contain the inserts from the cDNA library, were prepared they were packaged into the T-7 phage and amplified in E. coli strain BL21 in preparation for biopanning.

In vivo phage display selection: 100 μl (10⁸) of stock phage library was intravenously injected into mice (inferior vena cava for LS174T and portal vein for 1205LU) (FIGS. 2A and B). After a circulation time of 5 minutes mice were perfused via the heart and through the inferior vena cava until the perfusate was clear of blood. Liver, lung, kidney and brain were extracted and stored at −80 degrees centigrade. The organ of metastasis (lung or liver) was used to measure the phage titer which was then amplified in E. coli in preparation for the subsequent round of biopanning. A total of three to four rounds of biopanning were conducted. The organ-selected library obtained on completion, was used to randomly select plaques for sequencing. Individual clones selected from the target organ, were isolated, amplified and intravenously injected into mice to determine their degree of organ selectivity.

After the 1st round of biopanning, phage titer retained in the liver was only 0.03% and this rose by almost three logarithms (orders of magnitude) by the third round. By the fourth round of biopanning (FIG. 2D), 97% of the total number of retained phage from the third round, were retained in the liver, compared to just over 2% in the lungs and well under 1% in both the kidneys and brain (FIG. 2D). Alternatively, 76% of the total number of phage injected from the third round, were retained in the liver, indicating that after four rounds of biopanning, clones were selected which predominantly favored the liver.

Identification of Clones: The clones selected by 4 rounds of biopanning were plated. Sixty plaques were selected, amplified by PCR using primers from T7 and the nucleotide sequence determined. The number of clones sequenced depended upon the degeneracy of the library with respect to each clone. The sequences were then analyzed and the identity of the gene obtained by using the BLAST (n) program. Translated sequences started at the 5′ EcoRI site (GAATTC) at the 5′ junction between the T-7 select vector and the tumor cell gene. Any one of the three frames in which translation occurred was used as the authentic frame for translation. In the case of known genes, all three frames were run through the BLAST (p) program to determine the correct frame for translation.

For unknown genes, only frame translations, which were twenty amino acids or longer were used. The obtained amino acid sequences from various clones were grouped and analyzed by the CLUSTALW program (for multiple sequence alignment) in search of regions of homology among multiple clones.

Twenty five distinct clones were identified. Of the twenty five identified, seven were of unknown identity, fifteen were known to be either nuclear or cytoplasmic proteins, and one was associated with the cell membrane. Very surprisingly, none of the proteins identified were known to traverse the cell membrane. Of the twenty five different clones, LS42 was the most abundant being repeated seventeen times. A BLASTp search of the 151 amino acid insert shows that this peptide completely matched PA28alpha (alpha subunit) or IGUP 1-5111 from position 99 to 248. In the full length protein the first twenty four amino acids are indicated as the molecule's signal peptide allowing for secretion from the cytoplasm. Other than exhibiting 29% identity with β myosin heavy chain, this protein does not appear to be a member of a known family of proteins. However, it bears the cell adhesion motif RGD tripeptide. These results are both surprising and unexpected as a role for cell adhesion by PA28 alpha subunit has never been reported.

From a total of three separate experiments each comprising at least three rounds of in vivo selection, two of the most common five phage clones were found in all three experiments and three in at least two experiments. For example, the selected phage contained inserts coding for protein fragments of >100 amino acids and the most frequently observed gene fragments comprised: (1) the C-terminal portion of PA28alpha (e.g., amino acids 98 to 249 of GenBank Accession No. AAV38144), a cytoplasmic proteosome activator fragment that generates peptides for antigen presentation through MHC class 1, (2) the central portion of HNRNP A1 (e.g., amino acid residues 38 to 189 of GenBank Accession No. NP_{—}112420), a member of the large heterogeneous nuclear ribonucleoprotein family, and (3) an N-terminal region in a predicted protein of no known function, KIAA1407 (e.g., amino acids 96 to 293 of GenBank Accession No. BAA92645). PA28alpha (or PSME1) has been described as a cytosolic protein. In a FACS analysis with non-permeabilized LS174T cells, however, we found PA28alpha displayed on the surface of the cells, a finding that is reminiscent of a recent report describing the extracellular role in cancer invasion of another cytosolic protein, hsp90. The heterogeneous nuclear ribonucleoproteins comprise a large family of proteins involved with the intracellular processing of mRNA molecules and have been mostly found within the nucleus. Recently, however, it has been demonstrated that at least one of ribonucleoprotein family members, HNRNP M4 can function as a cell surface receptor for carcinoembryonic antigen (CEA). This extracellular role of HNRNP M4 as a receptor for the CEA cell attachment molecule would parallel the role of HNRNP A1 identified as a liver homing protein from colon cancer cells.

Immunohistochemistry

To demonstrate that the retention of clones in the liver was not due to nonspecific trapping but to direct binding to vascular cells, liver sections were probed with a T7 tag antibody and detected phage by immunohistochemistry. Localization of bacteriophage injected into mice was determined by immunohistochemistry analysis of brain, lung, liver and kidney tissue sections. After mice had been injected with bacteriophage and subsequently perfused, brain, lung, kidney and liver were removed and placed in 10% formaldehyde for 1-2 hours. Organs were then placed in 70% ethyl alcohol for at least two hours. Organs were embedded and sectioned and immunohistochemistry of tissue sections on glass slides was carded out. Briefly, embedding medium and formaldehyde were removed by pre-heating slides overnight at 55° C., followed by multiple treatments with xylene and ethanol. Sections were washed, blocked with 10% horse serum and after several more washes with phosphate buffered saline, (PBS), incubated with the primary antibody overnight. The following day sections were washed with PBS after which the biotinylated second antibody was added. Positive reactions were detected with avidin-biotin complex followed by incubation with DAB solution. Positive staining appeared as dark brown.

Northern Blot Analysis

Total RNA from cell lines was isolated with the RNA STAT-60 method (Tel-test, Friendswood, Tex.). RNA was separated and blotted as previously described (Fang et al., JBC, 1992, 267:25889-97). Blots were hybridized, washed and autoradiographed for 48 hrs with cDNA complementary to the gene which encodes for the 151 amino acid expression product for PA28alpha subunit. Glyceraldehyde-3-phosphate dehydrogenase (GAPDH) was used as a loading control.

In Situ Hybridization

To demonstrate that clones thus far identified in the mouse are significant in humans, phage inserts were used to prepare probes which were then used to probe human tissue arrays of both normal and cancer tissues. These tissue samples included breast, prostate, colon, brain and lymphatic system and both primary and metastatic tissue. In situ hybridizations were carried out as previously described (Stiletto et al., 2000). Briefly, deparaffinized sections of formalin-fixed tissues were treated at 37 C for 10 minutes with proteinase K and then washed twice with SSC. Slides were incubated overnight with respective oligonucleotides, in hybridization solution (50% formamide, 4×SSC, 1×Denhardt's solution, 5 mg/ml heat denatured salmon sperm DNA, 2.5 mg/ml yeast tRNA, 10% dextran sulfate). Slides were washed with 2×SSC for 30 minutes at room temperature, with 2×SSC/formamide at 50° C. and with 1×SSC at room temperature for five minutes. Anti-digoxigenin-alkaline phosphatase conjugate was used for immunological detection of bound probes. In the breast cancer tissues, the results showed that the gene was strongly expressed in three cases, medium expression in four cases and little or no expression in five cases.

Organ Homing by Individual Clones

After identification of the first twenty five clones from the liver-selective library, it was determined whether individual clones were capable of favoring the liver after being intravenously injected into mice. The nine most abundant clones were individually amplified and separately injected into no-tumor bearing mice. The nucleic acid sequences for the nine clones are provided in FIG. 2A-2C. Translations of the three reading frames for all nine clones are provided in FIG. 3A-3E. To minimize the possibility that clones were retained in the liver because of direct blood flow from portal vein injections, a selection of clones were also injected, in separate mice, through the inferior vena cava, and organ distribution of retained phage estimated.

Based on the selection method employed, the protein fragments identified define the liver homing domain of the respective genes and we found that the selected phage indeed bind tightly to the blood vessel lining of liver tissues when injected into animals and liver homing phage were detectable after tissue fixation and sectioning. Next we tested to what extent the binding of the selected phage to the liver vascular bed depends on the fact that we injected the library into the vena portae and thus the capillary bed of the liver is the first encountered by the phage particles. For this the selected phage particles were injected through an alternate route, i.e. into the vena cava and phage retained in different organs were then quantitated. From this systemic injection, phage particles first have to pass through the lung capillary bed before they reach the hepatic vessels. Empty phage (without a cDNA insert) showed no significant organ selectivity whereas the different liver homing phage displayed a >20-fold selectivity for the liver versus the lung vascular bed irrespective of the route of administration. Lung-homing phage selected from a different tumor cell library showed preference for the lungs. Thus, the selected homing genes will indeed seek out the vascular bed of the target organ irrespective of the route of administration and the first vascular bed encountered.

To standardize the phage numbers among different experiments, phage titers were represented as a proportion of the kidney titers within each experiment. Phage titers in the kidneys were selected for comparison since they were not in the direct circulatory pathway of phage injected either via the inferior vena cava or portal vein. This new number was then used to calculate the liver to lung ratio which was finally used as a measure of liver selectivity. A ratio of one indicates that the clone was distributed equally between the lungs and liver. Injection of the control, wild type T7 phage via the inferior vena cava resulted in a liver to lung ratio of just over one. A slight preference for the liver (two fold) was seen when the route of injection was the portal vein.

Despite greater liver to lung ratios when the route of delivery was the portal vein as compared to the inferior vena cava, there was a clear preference for the liver in the clones tested (at least fourteen fold), in comparison to the numbers seen with the control, empty T7 phage. While some of the repeat experiments for individual clones showed variable numbers (e.g. 29 fold and 270 fold for PA28alpha), all ratios were well above those seen for control phage. Thus, the clones selected for intravenous injection are mostly liver-selective. As further confirmation of the selectivity of these clones, the only clone injected individually whose sequence was outside the open reading frame, JN42, displayed a very weak level of selectivity (two fold), comparable to that of the control phage.

To demonstrate that the method described herein is generally applicable to all possible metastatic tissues, a cell line known to metastasize to organs besides the liver was tested. One of the tumor cell lines used was the human melanoma cell line 1205LU which predominantly metastasizes to the lungs. Using the same principal as that previously described for LS174T, a cDNA library was generated from 1205LU which was then spliced into the T7 phage. After biopanning this library in mice, individual clones were tested for their ability to preferentially home to the lungs. In spite of skepticism in the literature about being able to show selection in the lungs due to their high perfusion, a preference of the RFC2 clone for the lugs over the liver by 2.9 fold was shown (see Pasqualini and Ruoslahti Nature 38:1996, 364-366).

Identification of genes that predict and potentially drive metastasis in patients with gastrointestinal carcinoma can be a key concern for diagnostics and therapy.

Patients with different cancers as well as a secreted fibroblast growth factor binding protein (FGF-BP) that is upregulated early in the progression of colon cancer, i.e. dysplasia. We report the mRNA expression of five of the novel metastasis genes from phage display as well as PTN and FGFBP using a series of 39 tissue microarrays representing cancers of the pancreas (n=106), ampulla (n=54), bile duct (n=40), colon (n=37) and liver metastases from colon cancers (n=35). Each of these tumors was represented by several cores on the arrays (mean 4.8±2.6) and 708 cores of 22 different reference tissues were used as controls. Staining for mRNA was performed by in situ hybridization (ISH) with digoxigenin-labeled antisense mRNA probes.

Corresponding controls were performed with sense probes. Staining was evaluated without prior knowledge of the clinical data. Each core was classified according to staining intensity and frequency and tumor cases were classified by percentage of positively staining cancer cells into 6 groups from negative to highly positive.

Results show a distinct frequency and intensity of gene expression in most of the primary lesions (56.2%-92.2%) and very high expression in the liver metastases (69.09-100%). Expression of all of the genes was low in the pancreas non-adeno-carcinoma (25.8-27.3%) and in the according non-neoplastic reference tissues (0%-25%).

A subsequent comparison of patient cases with and without known metastasis showed highly significant increases in expression levels and frequency of the phage-display derived metastasis genes and of PTN for the cases with metastasis. Tumors with known metastasis typically showed >75% positive tumor cells. Tumors with low or no expression (<25%) were typically without metastasis (all p-values <0.05). No significant correlations were found for FGF-BP. We conclude that genes discovered by phage display and PTN can serve to distinguish amongst GI cancers with different stage and outcome.

To evaluate the significance of for human cancers, a series of normal and diseased human tissues were examined for expression of these genes. Tissue samples from colon cancer metastases to the liver as well as primary colon cancers that had metastasized at the time of diagnosis were evaluated in parallel with invasive colon cancers without known metastasis and normal colon tissues. In addition, breast cancer samples were used as a control due to a relatively low propensity of breast cancer for initial metastasis to the liver. All of these analyses were carried out with tissue microarrays and included >1,200 tissue samples for each of the genes studied. In situ hybridization for mRNA expression was used for detection of gene expression. A good signal-to-noise ratio between normal colon or normal liver and invasive colon cancer and liver metastases of colon cancer was found for all three of the genes probed. In serial sections approximately 60% of invasive colon cancer without metastases and >95% of the samples from metastatic cancers showed expression of all three of the liver homing genes whereas less than 10% of normal colon tissues showed any detectable signal. A significant difference in the frequency of expression was observed between cancer and normal tissues and between non-metastatic and metastatic colon cancer samples. After a semi-quantitative evaluation of expression levels in the serial sections we then calculated a cumulative expression score of the homing genes for each of the tissues. This cumulative expression score of the homing genes revealed highly significant (p<0.001) and striking differences between normal colon tissues, primary colon cancers without and with metastasis at the time of diagnosis and liver metastases of colon cancer. The cumulative level of expression of homing genes in metastatic disease was shifted to the highest levels, normal tissues ranked at the low end and non-metastatic disease in between. Much in contrast with this, expression of a gene that is induced during initiation of colon epithelial transformation, FGF-BP, showed no discernible differences amongst the metastatic and non-metastatic colon cancer samples. Furthermore, invasive breast cancers showed an overall low frequency of expression of the liver homing genes and no difference with respect to the metastatic status of the tumors. This latter finding is in line with the distinct metastasis pattern of breast and colon cancer and we conclude that expression of the liver homing genes in primary colon cancer is predictive of a high likelihood of its metastatic spread.

### Example 2

### Use of the Selected Phage to Inhibit Retention of Tumor Cells in the Liver

A series of experiments was designed to test whether the homing genes identified herein, such as for example, PA28alpha, HNRNPA1, and/or KIAA1407 are rate-limiting for tumor cell attachment to endothelial cells and tumor cell seeding into the liver. To test this, we used the selected phage as blocking agents. It was reasoned that the respective homing domains presented by the phage would bind to a target site of tumor cells on the endothelial cells or the vessel wall and thus could reduce tumor cell attachment. In an in vitro set of experiments, the LS174T tumor cells were mixed with control phage or the selected liver homing phage and the effect on tumor cell attachment to an endothelial cell layer was assessed. The liver homing phage were able to prevent attachment of tumor cells to cultured endothelial cells and 2- to 3-fold more tumor cells were found unattached in the presence of the liver homing phage relative to the control phage. This data supports the notion of a surface interaction mediated by the homing proteins during the tumor cell attachment to the endothelial layer. In a parallel animal experiment we then evaluated the ability of the selected phage to impact on the homing of tumor cells to the liver vascular bed. For this, the LS174T tumor cells were mixed with liver-homing phage or with unselected phage and injected into the spleen as a conduit to the liver via the vena portae. Tumor cells retained by the liver vascular bed were by measuring the amount of luciferase that had been transiently transfected before the injection. After 10 minutes, the number of tumor cells seeding into the liver vascular bed was assessed by luciferase assay. Liver horning phage significantly inhibited retention of the LS174T cells in the liver. This was true for the composite of the 1:Q selected phage as well as for an individual phage containing the PA28alpha liver homing domain as an insert.

Liver retention of LS174T cells injected without any phage was comparable to the retention of LS174T cells coinjected with the unselected control phage. Also, HEK293T epithelial cells used as a control were not inhibited by the liver homing phage. A significant portion of seeding of the LS174T tumor cells to the liver requires the recognition of the binding sites of their homing proteins.

Without being bound by theory, it appears that the identified genes encoding targeting domains of PA28alpha, HNRNP A1 and KIAA1407 did not contain canonical signatures of cell/cell interaction or known cell surface location. It appears counterintuitive that nuclear or cytosolic proteins such as PA28alpha or HNRNP A1 would be homing proteins. The finding that HNRNP M4 as a ligand for CEA (Bajenova et al, 2001, I. Biol. Chem. 276:31067 and Bajenova et al, 2003, Exp Cell Res 291:228), extracellular hsp90 modulating cancer invasion (Eustace et al., 2004, Nat. Cell Biol 6:507-14 and Picard et al., 2004 Nat. Cell Biol 6:479) and the finding that PA28alpha on the surface of tumor cells by FACS analysis supports the fact that intracellular proteins can be functional as cell surface ligands. The finding that protein fragments of PA28alpha, HNRNP A1 and KIAA1407 as liver homing domains is corroborated independently by the blocking of tumor cell attachment to an endothelial layer in vitro and by the inhibition of homing of tumor cells to the liver in vivo. All three of PA28alpha, HNRNP A1 and KIAA1407 show a high frequency of expression in metastatic colon cancer. This finding provides an opportunity for imaging of metastasis comprising the use of PA28alpha, HNRNP A1 and KIAA1407, fragments thereof, in particular the fragments disclosed herein in the figures and/or polynucleotides encoding them, as well as therapeutic targeting, such as by the use of compositions comprising a targeting domain, such as a domain of PA28alpha, HNRNP A1 and/or KIAA1407, linked to a therapeutic agent. Such targeting domains linked to a therapeutic agent can be administered to a subject in need, such as a subject at risk for or subject to cancer, with or without metastasis.

### Methods:

Quantitation of Cell Surface PA28 alpha Expression by FACS

LS174T cells were treated with 0.02% Na₂EDTA washed twice in PBS and then incubated with 0.4 ml of 1:50 dilution of anti-paxillin (UBI) or anti-PA28alpha antibody (Biotrend, Koln, Germany) for 15 minutes at room temperature. In one subset of experiments, cells were permeabilized by inclusion of 10% Triton-X during each incubation step. After three cold PB washes the cells were incubated with 0.4 ml of a 1:200 dilution of rabbit anti-mouse FITC for 15 minutes at 40° C. After fixation in 3% paraformaldehyde, the cells were diluted to a concentration of 0.3% paraformaldehyde and the fluorescent intensity was measured by FACS. Paxillin served as a control for the permeability and surface location of PA28alpha in the cells.

Tissue Arrays and In Situ Hybridization for mRNA Expression

In situ hybridization protocols were as described for other probes (Ray et al., 2003, Cancer Res. 63, 8085-9 and Schulte et al., 2000, Oncogene 19, 3988-98). T7 inserts were amplified by PCR and cloned into the TOPO TA cloning vector (Invitrogen; Carlsbad, Calif.) from which Digoxigenin-labelled RNA riboprobes were transcribed using Sp6 polymerase. In brief, slides were deparaffinized and rehydrated with xylene, ethanol and water. Proteins were digested with proteinase K at 37° C. for 10 minutes and acetylated with acetic anhydride for 15 minutes. The RNA-probe was then mixed with hybridization solution (Sigma-Aldrich, St. Louis, Mo.; 1.5 ng/1.0 μl) and incubated with the respective tissues for 16 hrs at 42° C. Unbound RNA-probe was digested with RNase A (10 μg/ml, Roche) for 15 minutes. Highly stringent washing, re-fixation and cross-linking was performed by Formamide/SSC 2× (1:1), SSC 1× and SSC 0.5×, each for 10 minutes at 52° C. After blocking with a 2% horse serum for 30 minutes, a 1:250 solution of alkaline-phosophatase (AP) tagged anti-digoxigenin antibody fragments (Roche) in buffer (100 mM Tris-HCl pH 7.5 and 150 mM NaCl) was incubated with the tissue for 16 hrs at 4° C. The staining solution of nitroblue tetrazolium/5-bromo-4-chloro-3-indolylphosphate (Roche) was then applied and the reaction was terminated once sufficient staining was observed, using 10 mM Tris-HCl pH 8.0, 1 mM EDTA for 10-min. Finally, slides were air dried and mounted.

### Homing of Tumor Cells to the Liver

To assess the ability of selected phage to affect seeding of LS174T cells to the liver, 10⁷ LS174T cells were transiently transfected with a 7 μg CMV luciferase construct/20 μl FUGENE 6 transfection reagent (Roche) mixture. Twenty four hours later the cells were harvested and luciferase expression determined from an aliquot of the LS174T cells employing a Firefly Luciferase Reporter System Assay (Promega, Madison, Wis.) and a Lumat LB9501 luminometer (Berthold, Bad Wildbad, Germany). 10⁵ of the transiently transfected LS174T cells were then injected via the hepatic portal circulation with 10⁷ unselected phage, round four selected phage or PA28alpha containing phage. Following a five minute circulation period, the liver was harvested and Luciferase activity was detected in 100 of liver μl of liver homogenate to derive the number of tumor cells retained.

### Example 3

### Determination of Survival Rates

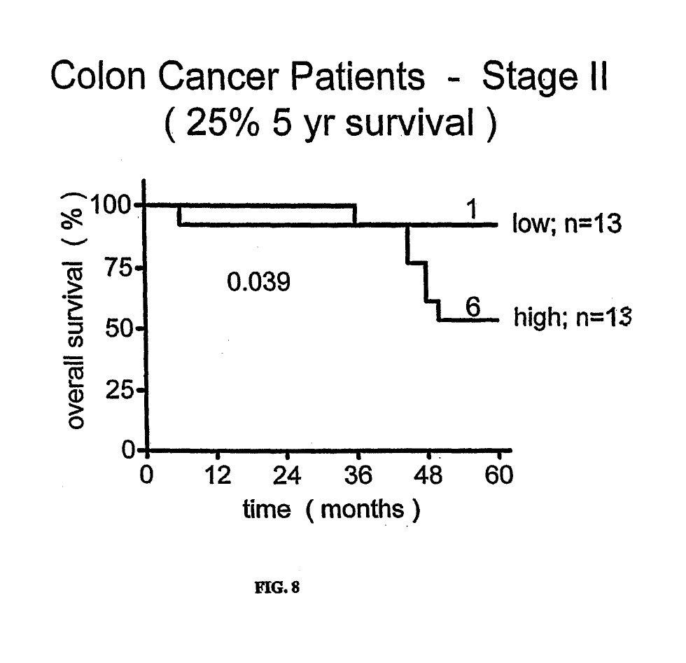

Tissue samples/arrays shown and used in FIGS. 6A-6B and FIGS. 7A-7D were obtained from Clinomics Biosciences, Inc. Frederich, Md. mRNA encoding each of the targeting portions of PA28alpha, HNRNP A1 and KIAA1407 were used in in situ hybridizations against the tissue arrays, as described in Example 2. The figures show cumulative scoring of PA28alpha, HNRNP A1 and KIAA1407 together (6A) as well as individual scoring (6B) as correlated with survival. FIGS. 7A-7D compare colon cancer results with breast cancer results. The five year mortality rate for Stage II colon cancer is 25%, that is, 1 out of 4 individuals with Stage II cancer will die by the end of five years. FIG. 8 shows that in a total number of 26 subjects diagnosed with Stage II colon cancer, 6 out of 7 had a “high” cumulative score (that is greater than about 4 or 5 out of 9, with a high for each targeting domain being 3) of the measurements of PA28alpha, HNRNP A1 and KIAA1407.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for determining the probability of survival in an individual diagnosed with colon cancer, comprising, measuring the presence of elevated levels of one or more of the following:
(a) a polynucleotide disclosed herein in FIGS. 2A-2C, or a complement thereof;
(b) a polynucleotide encoding any one of the polypeptides disclosed herein in FIGS. 3A-3E, or a complement thereof;
(c) a polynucleotide encoding any one of the polypeptides disclosed herein in FIGS. 10 through 13, or a complement thereof;
(d) a polypeptide disclosed herein in FIGS. 3A-3E, and/or a fragment thereof; or
(e) a polypeptide disclosed herein in FIGS. 10-13, or fragment thereof,
wherein the presence of elevated levels of any one of (a) through (e) as compared to a control, is correlated with a decrease in the probability of survival.

2. The method of claim 1 comprising measuring the levels of two or more of PA28alpha, HNRNPA1, KIAA1407, or polynucleotides encoding them, wherein the presence of elevated levels of two or more of PA28alpha, HNRNPA1, KIAA1407, or polynucleotides encoding them is correlated with a decrease in the probability of survival.

3. The method of claim 1 comprising measuring the cumulative levels of PA28alpha, HNRNPA1, and KIAA1407, or polynucleotides encoding them, wherein the presence of cumulative elevated levels of PA28alpha, HNRNPA1, and KIAA1407, or polynucleotides encoding them, is correlated with a decrease in the probability of survival.

4. The method of claim 1 wherein the probability of survival is characterized by a survival rate of five years.

5. A method of predicting the likelihood that a primary colon cancer in an individual will metastasize to the liver of the individual, comprising analyzing the primary colon cancer for at least one of the following:
(a) a liver targeting gene or a complement thereof;
(b) a protein encoded by a liver targeting gene;
(c) a homing domain of a liver targeting gene product;
(d) nucleic acid encoding a homing domain of (c) or a complement of the nucleic acid encoding a homing domain of (c); and
(e) mRNA of a homing domain of (c) or the complement thereof,
wherein if at least one of (a)-(e) is present in the primary colon cancer, there is an increased likelihood the primary colon cancer will metastasize to the liver of the individual than if at least one of (a)-(e) is present at a lower level relative to an appropriate control.

6. The method of claim 5, wherein:
(a) is selected from: (i) a gene encoding PA28 alpha; (ii) a gene encoding HNRNP A1; and (iii) a gene encoding KIAA1407;
(b) is selected from: (i) PA28 alpha; (ii) HNRNP A1; (iii) KIAA1407; (iv) PA28 alpha mRNA; (v) HNRNP A1 mRNA; and (vi) KIAA1407 mRNA;
(c) is selected from: (i) a homing domain of PA28 alpha; (ii) a homing domain of HNRNP A1; and (iii) a homing domain of KIAA1407;
(d) is selected from: (i) nucleic acid encoding a homing domain of PA28 alpha; (ii) nucleic acid encoding a homing domain of HNRNP A1; and (iii) nucleic acid encoding a homing domain of KIAA1407; and
(e) is selected from (i) mRNA of a homing domain of PA28 alpha; (ii) mRNA of a homing domain of HNRNP A1; and (iii) mRNA of a homing domain of KIAA1407.

7. The method of claim 5, wherein (c) is a C-terminal portion of PA28 alpha; a central portion of HNRNP A1, or an N-terminal region of KIAA1407, or a combination of two or three of the homing domains.

8. The method of claim 7, wherein the C-terminal portion of PA28 alpha is encoded by SEQ ID NO: 7; the central portion of HNRNP A1 is encoded by SEQ ID NO: 5; and the N-terminal region of KIAA1407 is encoded by SEQ ID NO: 8.

9. The method of claim 5, wherein the primary colon cancer is analyzed for one or more of the following: (i) PA28 alpha; (ii) a homing domain of PA28 alpha; (iii) HNRNP A1; (iv) a homing domain of HNRNP A1; (v) KIAA1407 and (vi) a homing domain of KIAA1407.

10. The method of claim 9, wherein at least one antibody that specifically recognizes (i) PA28 alpha; (ii) a homing domain of PA28 alpha; (iii) HNRNP A1; (iv) a homing domain of HNRNP A1; (v) KIAA1407 or (vi) a homing domain of KIAA1407 is used to analyze the primary colon cancer.

11. The method of claim 9, wherein a combination comprising at least two of the following antibodies: (a) an antibody that specifically recognizes PA28 alpha; (b) an antibody that specifically recognizes HNRNP A1; and (c) an antibody that specifically recognizes KIAA1407 is used to analyze the primary colon cancer.

12. The method of claim 11, wherein a combination that comprises the following: (a) an antibody that specifically recognizes PA28 alpha; (b) an antibody that specifically recognizes HNRNP A1; and (c) an antibody that specifically recognizes KIAA1407 is used to analyze the primary colon cancer.

13. The method of claim 9, wherein a combination comprising at least two of the following antibodies: (a) an antibody that specifically recognizes a homing domain of PA28 alpha; (b) an antibody that specifically recognizes a homing domain of HNRNP A1; and (c) an antibody that specifically recognizes a homing domain of KIAA1407 is used to analyze the primary colon cancer.

14. The method of claim 13, wherein a combination that comprises the following: (a) an antibody that specifically recognizes a homing domain of PA28 alpha; (b) an antibody that specifically recognizes a homing domain of HNRNP A1; and (c) an antibody that specifically recognizes a homing domain of KIAA1407 is used to analyze the primary colon cancer.

15. The method of claim 5, wherein the primary colon cancer is analyzed for one or more of the following: (a) a gene encoding PA28 alpha or a complement thereof; (b) a gene encoding HNRNP A1 or a complement thereof; (c) a gene encoding KIAA1407 or a complement thereof; (d) nucleic acid encoding a homing domain of PA28 alpha or a complement thereof; (e) nucleic acid encoding a homing domain of HNRNP A1 or a complement thereof; (f) nucleic acid encoding a homing domain of KIAA1407 or a complement thereof; (g) mRNA of a homing domain of PA28 alpha or a complement thereof; (ii) mRNA of a homing domain of HNRNP A1 or a complement thereof; and (iii) mRNA of a homing domain of KIAA1407 or a complement thereof.

16. The method of claim 15, wherein the primary colon cancer is analyzed for mRNA expression of a liver targeting gene or mRNA expression of a homing domain of a liver targeting gene.

17. The method of claim 16, wherein the primary colon cancer is analyzed by in situ hybridization for mRNA expression of at least one of the following:
(a) a gene encoding PA28 alpha or a complement thereof;
(b) a gene encoding HNRNP A1 or a complement thereof;
(c) a gene encoding KIAA1407 or a complement thereof;
(d) nucleic acid encoding a homing domain of PA28 alpha or a complement thereof;
(e) nucleic acid encoding a homing domain of HNRNP A1 or a complement thereof;
(f) nucleic acid encoding a homing domain of KIAA1407 or a complement thereof;
(g) mRNA of a homing domain of PA28 alpha or a complement thereof;
(h) mRNA of a homing domain of HNRNP A1 or a complement thereof; and
(i) mRNA of a homing domain of KIAA1407 or a complement thereof.

18. A method of predicting or aiding in the prediction that an individual with colon cancer will survive at least one year after diagnosis of a primary colon cancer, comprising:
(a) analyzing a colon cancer sample obtained from the individual for expression of at least one liver targeting gene, thereby producing a sample score for the individual;
(b) comparing the sample score for the individual with results of corresponding analysis, referred to as a control score, of colon cancer samples obtained from individuals who survived at least one year after diagnosis of a primary colon cancer,
wherein if the sample score for the individual is greater than the control score, the individual with colon cancer is less likely to survive at least one year after diagnosis than if the sample score for the individual is less than the control score.

19. The method of claim 18, wherein the colon cancer sample is selected from a sample from a primary colon cancer, a sample from a tissue to which the primary colon cancer has metastasized, a body fluid.

20. The method of claim 19, wherein in (a), the colon cancer sample is analyzed for at least one liver targeting gene; at least one homing domain of a liver targeting gene; at least one nucleic acid encoding a homing domain of a liver targeting gene; or at least one mRNA of a homing domain of a liver targeting gene, thereby producing a sample score for the individual and in (b) the sample score is compared with results of corresponding analysis of colon cancer samples obtained from individuals who survived at least one year after diagnosis of a primary colon cancer.

21. The method of claim 20, wherein:
(a) the liver targeting gene is a gene encoding PA28 alpha or a complement thereof; a gene encoding HNRNP A1 or a complement thereof; a gene encoding KIAA1407 or a complement thereof;
(b) the homing domain of a liver targeting gene is a homing domain of PA28 alpha; a homing domain of HNRNP A1; or a homing domain of KIAA1407;
(c) the nucleic acid encoding a homing domain of a liver targeting gene is a nucleic acid encoding a homing domain of PA28 alpha; a nucleic acid encoding a homing domain of HNRNP A1; or nucleic acid encoding a homing domain of KIAA1407; and
(d) the mRNA of a homing domain of a liver targeting gene is mRNA of a homing domain of PA28 alpha; mRNA of a homing domain of HNRNP A1; or mRNA of a homing domain of KIAA1407.

22. The method of claim 19, wherein in (a), the colon cancer sample is analyzed for at least two liver targeting genes; a homing domain of at least two liver targeting gene products; at least two nucleic acids, each of which encodes a homing domain of a different liver targeting gene; or at least two mRNAs, each of which is a homing domain of a different liver targeting gene product, thereby producing a sample score for the individual and in (b) the sample score is compared with results of corresponding analysis of colon cancer samples obtained from individuals who survived at least one year after diagnosis of a primary colon cancer.

23. The method of claim 19, wherein in (a), the colon cancer sample is analyzed for at least three liver targeting genes; a homing domain of at least three liver targeting gene products; at least three nucleic acids, each of which encodes a homing domain of a different liver targeting gene; or at least three mRNAs, each of which is a homing domain of a different liver targeting gene product, thereby producing a sample score for the individual and in (b) the sample score is compared with results of corresponding analysis of colon cancer samples obtained from individuals who survived at least one year after diagnosis of a primary colon cancer.

24. The method of claim 19, comprising analyzing a colon cancer sample obtained from the individual for expression of at least two of the following liver targeting genes: PA28 alpha; HNRNP A1; and KIAA1407, thereby producing a sample score for the individual and comparing the sample score for the individual with results of corresponding analysis, referred to as a control score, of colon cancer samples obtained from individuals who survived at least one year after diagnosis of a primary colon cancer.

25. The method of claim 19, comprising analyzing a colon cancer sample obtained from the individual for expression of PA28 alpha; HNRNP A1; and KIAA1407, thereby producing a sample score for the individual and comparing the sample score for the individual with results of corresponding analysis, referred to as a control score, of colon cancer samples obtained from individuals who survived at least one year after diagnosis of a primary colon cancer.

26. The method of claim 18, wherein the method is for predicting or aiding in predicting that the individual will survive at least 5 years after diagnosis.

27. A method of targeting an agent to colon cancer in an individual, comprising administering to an individual in need thereof an effective amount of a composition comprising a homing domain of a liver targeting polypeptide and the agent to be targeted to colon cancer in the individual, whereby the agent is targeted to the colon cancer.

28. The method of claim 27, wherein the agent to be targeted to colon cancer is a therapeutic drug or a detection agent and the colon cancer is a primary colon cancer or colon cancer that has metastasized to the liver.

29. The method of claim 28, wherein the agent is a therapeutic drug selected from ricin, a radioisotope, a chemotherapeutic and radiosensitizing agent, an anti-angiogenesis agent, an anti-motility agent, and an immunomodulatory agent.

30. The method of claim 29, wherein the agent is a detection agent selected from radioisotopes, dyes, fluorescent molecules, and pigments.

31. An isolated antibody which specifically binds PA28alpha, HNRNPA1 or KIAA1407.

32. The isolated antibody of claim 31 which is a monoclonal antibody.

33. A composition comprising (a) a homing domain of a liver targeting polypeptide and (b) an agent to be targeted to colon cancer in an individual.

34. The composition of claim 33, wherein the homing domain is a homing domain of PA28alpha; a homing domain of HNRNPA1; or a homing domain of KIAA1407, and the agent to be targeted to colon cancer is a therapeutic drug or a detection agent.

35. The composition of claim 34, which is a pharmaceutical composition further comprising a pharmaceutically acceptable carrier.

36. The pharmaceutical composition of claim 34, wherein the therapeutic drug is selected from ricin, a radioisotope, a chemotherapeutic and radiosensitizing agent, an anti-angiogenesis agent, an anti-motility agent, and an immunomodulatory agent.

37. The composition of claim 34, wherein the agent to be targeted is a detection agent which is a radioisotope, a dye, a fluorescent molecule, a pigment.
